(19) United States
(12) Patent Application Publication
(10) Pub. No.: US 2026/0207107 A1
(43) Pub. Date: Jul. 23, 2026

(54) ADHESIVE HYDROGEL-INTEGRATED BIOELECTRONIC DEVICE

(71) Applicants: Chinese Institute for Brain Research, Beijing, Beijing (CN); Institute of Chemistry, Chinese Academy of Sciences, Beijing (CN)

(72) Inventors: Ting WU, Beijing (CN); Liju XU, Beijing (CN); Lin CHEN, Beijing (CN); Dong QIU, Beijing (CN)

(21) Appl. No.: 19/644,761

(22) Filed: Apr. 10, 2026

(51) Int. Cl. A61B 5/293; A61B 5/00; A61B 5/259; C09J 9/02

(52) U.S. Cl. CPC A61B 5/293; A61B 5/259; A61B 5/6847; A61B 5/6868; C09J 9/02; A61B 2562/046; A61B 2562/12; A61B 2562/164; A61B 2562/166; A61B 2562/18; C09J 2301/414; C09J 2483/00

(57) ABSTRACT

An implantable bioelectronic device, comprising: a flexible insulating substrate; a conductive circuit layer disposed on the flexible insulating substrate and comprising an array of electrodes comprising one or more electrodes; an insulating encapsulation layer encapsulating the conductive circuit layer and comprising an opening, wherein the electrodes are exposed through the opening; and a conductive adhesive interface layer disposed on the insulating encapsulation layer and comprising an exposed surface and a filling portion that seals the opening, wherein the exposed surface is electrically connected to the electrodes through the filling portion, the conductive adhesive interface layer comprises a hydrophilic polymer and a hydrophobic polymer, and wherein the hydrophobic polymer is formed by polymerization of 3-(trimethoxysilyl)propyl methacrylate (TPM) monomers.

### FIELD OF THE INVENTION

The invention relates generally to an implantable bioelectronic device, a human-machine interface system comprising the same and applications thereof.

### BACKGROUND OF THE INVENTION

Electrocorticography (ECoG) is an invasive neural monitoring technique that requires surgical placement of electrode grids or strips directly on the surface of the cerebral cortex (gray matter) to record cortical electrical activity. μECoG arrays are increasingly used in functional cortical mapping, epilepsy monitoring, and brain-computer interfaces (BCIs), owing to its superior spatiotemporal resolution compared to traditional electroencephalography (EEG). However, the long-term reliability of μECoG devices, such as μECoG arrays, is compromised by adverse tissue responses at the electrode-tissue interface. Despite their flexibility, the substantial mechanical modulus mismatch between μECoG arrays (Young's modulus E>1 GPa) and soft brain tissue (E <30 kPa) induces chronic inflammatory responses, including glial cell activation, progressive gliosis, and fibrotic capsule formation. These adverse tissue responses are believed to cause impedance fluctuations, deteriorate signal quality, and potentially lead to device dysfunction. Additionally, because μECoG devices lack intrinsic adhesive properties, microscale displacement caused by bodily motion and cerebrospinal fluid (CSF) dynamics could introduce instability in high spatial resolution recording. External anchoring methods, such as sutures or adhesive glues, are required but may exacerbate biocompatibility concerns.

Recent advances in hydrogel-integrated bioelectronics offer promising strategies to address critical challenges, including the improvement of mechanical modulus, conductivity, durability, and adhesiveness, which are essential for reliable and long-term biointerfacing applications. Approaches such as bio-inspired adhesive chemistries, hierarchical structural design, and advanced crosslinking mechanisms have demonstrated significant progress in enhancing hydrogel performance across various biointerfaces, including cardiac, nerve, skin, subcutaneous, and neuromuscular systems. However, translating these successes into neural interfaces requires more than simple adaptation because of the unique physiological and mechanical complexities of the brain tissue. Emerging neural-specific designs, such as highly conductive carrageenan-interpenetrated PDA-polyacryl-amide hydrogel-integrated μECoG arrays, exhibit immune-invasive capabilities and provide reliable signal transduction (X. Wang, et al., Matter 2022, 5, 1204-1223). Similarly, non-deformable hydrogel patches have been shown to enhance the recording stability of μECoG arrays for a week, while minimizing fibrotic tissue responses (W. Li, et al., Adv. Funct. Mater. 2025, 2422717). Additionally, catechol-conjugated alginate hydrogel cortex adhesive sensors have demonstrated stable epidural recording during transcranial focused ultrasound neurostimulation for nearly half a year (S. Lee, T et al., Nat. Electron. 2024, 7, 800-814). Despite these advances, these systems mainly rely on chemical modifications, such as catechols and N-hydroxysuccinimide for tissue adhesion. While these approaches ensure robust and permanent adhesion, they raise concerns about chemical contamination and potential damage during implant removal. In contrast, hydrogel-integrated arrays that rely solely on physical hydrogen bonding mechanisms offer reversible attachment and mitigate chemical risks (S. Oribe, et al., Sci. Rep. 2019, 9, 13379; D. W. Kim, et al., Adv. Mater. 2022, 34, 2105338). However, their long-term subdural implantation remains underexplored, particularly in terms of the evaluation of signal stability during the progression of neuroinflammation and fibrotic tissue formation. Moreover, previous studies have often overlooked the spatial decoupling effect by interposing a functional hydrogel layer between the electrodes and neural tissue, which introduces signal attenuation governed by the classic volume conduction model. Suboptimal parameter designs for hydrogel thickness and conductivity may compromise the electrical fidelity of low-amplitude neural recordings. Bridging these gaps requires innovative approaches to optimize material properties, adhesion mechanisms, and long-term in vivo biocompatibility and stability evaluation.

### SUMMARY OF THE INVENTION

Existing flexible μECoG electrodes face several problems during long-term implantation, including weak hydrogel-electrode interface bonding, insufficient hydrogel-brain tissue interface adhesion, and poor anti-swelling and mechanical properties of the hydrogel body. To address at least one of the problems, an adhesive hydrogel-integrated μECoG (aGel-μECoG) neural interface that mitigates tissue responses and ensures long-term signal stability is provided. In some embodiments, the present disclosure provides a hydrogel-integrated neural interface that meets the following requirements: i) robust tissue adhesion with reversible bonding to prevent cortical damage during explantation; ii) an electrically transparent hydrogel coating with spatiotemporal resolution comparable to that of its host device; and iii) minimized neuroinflammation and fibrotic capsule formation for sustained performance in vivo.

To meet these requirements, a first illustrative aspect of the invention provides an implantable bioelectronic device, comprising: a flexible insulating substrate; a conductive circuit layer disposed on the flexible insulating substrate and comprising an array of electrodes comprising one or more electrodes; an insulating encapsulation layer encapsulating the conductive circuit layer and comprising an opening, wherein the electrodes are exposed through the opening; and a conductive adhesive interface layer disposed on the insulating encapsulation layer and comprising an exposed surface and a filling portion that extends into the opening, wherein the exposed surface is electrically connected to the electrodes through the filling portion, the conductive adhesive interface layer comprises a hydrophilic polymer and a hydrophobic polymer, and wherein the hydrophobic polymer comprises a polymerized network of silane-based monomers.

In some embodiments, the silane-based monomers each independently has a general formula: Rₐ(CH₂)ₙSi(OR_{b})₃, wherein Rₐ represents a group comprising a polymerizable functional group selected from the group consisting of an addition-polymerizable functional group, a condensation-polymerizable functional group, and a ring-opening polymerizable functional group; R_{b}s are the same or different, and are each independently selected from the group consisting of C₁₋₁₂ alkyl, and —C₁₋₁₂ alkylene-O—C₁₋₁₂ alkyl; and n is an integer of 0 to 12, preferably, n is an integer of 1 to 8, more preferably, n is an integer of 1 to 3.

In some embodiments, the silane-based monomers comprise: 3-(trimethoxysilyl)propyl methacrylate (TPM), vinyltrimethoxysilane (KH171), vinyltris (β-methoxyethoxy) silane (KH172), vinyltriethoxysilane (KH151), glycidoxypropyltrimethoxysilane, or combinations thereof. Preferably, the silane-based monomers are 3-(trimethoxysilyl)propyl methacrylate.

In some embodiments, the addition-polymerizable functional group is a carbon-carbon double bond or a carbon-carbon triple bond.

In some embodiments, the condensation-polymerizable functional group is an amino group, a carboxyl group, or a hydroxyl group.

In some embodiments, the ring-opening polymerizable functional group is a 3-20 membered epoxy group, a 4-20 membered lactone group, or a 4-20 membered lactam group.

In some embodiments, Ra is selected from at least one of vinyl, ethynyl, amino, methacryloyloxy, hydroxy, and glycidyl ether. Preferably, Rₐ is methacryloyloxy or glycidyl ether.

In some embodiments, R_{b}s are the same, and are each independently selected from the group consisting of C₁₋₆ alkyl, and —C₁₋₆ alkylene-O—C₁₋₆ alkyl.

In some embodiments, the hydrophilic polymer has a plurality of hydrophilic groups and is configured such that, when the conductive adhesive interface layer is put in contact with a moist biological tissue, the hydrophilic groups form multiple hydrogen bonds with nitrogen-containing or oxygen-containing functional groups of the biological tissue.

In some embodiments, the hydrophilic groups comprise one or more hydroxyl groups, one or more carboxyl groups, one or more amino groups, and/or one or more sulfonic acid groups.

In some embodiments, the hydrophilic polymer is selected from the group consisting of: polyvinyl alcohol (PVA), polyethylene oxide, hydrophilic polyurethane, hydrophilic epoxy resin, polyacrylamide, polyethylene glycol, poly(hydroxyethyl methacrylate), poly(hydroxyethyl acrylate), polyacrylic acid, copolymers thereof, and combinations thereof.

In some embodiments, wherein the number average molecular weight of the hydrophilic polymer is 1000 to 500000, preferably, is 5000 to 100000, such as 5000, 50000, 100000.

In some embodiments, the conductive adhesive interface layer is formed by in-situ polymerization of a composition comprising the hydrophilic polymer or monomers thereof and the silane-based monomers, particularly within the opening; preferably, the composition comprises 2-15 parts by weight of the hydrophilic polymer or monomers thereof, 0.1-10 parts by weight of the silane-based monomers, and 0.0001-0.5 parts by weight of an initiator.

In some embodiments, the mass ratio of the hydrophilic polymer to the hydrophobic polymer ranges from 3.5:1 to 17:1, including specific ratios such as about 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, 15:1, 16.5:1, or 17:1, preferably, the mass ratio is ranging from 4:1 to 12:1, more preferably, the mass ratio is about 7:1.

In some embodiments, the conductive adhesive interface layer is in a form of hydrogel. In some embodiments, the conductive adhesive interface layer is in a form of dehydrated hydrogel.

In some embodiments, the conductive adhesive interface layer is a hydrogel and has a thickness of about 1~100 μm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 μm or any number therebetween, preferably, the conductive adhesive interface layer has a thickness of 10 μm. If the conductive adhesive interface layer is too thin, the crosslinking between the monomers may not be successful; and if it is too thick, the quality of the signal may be affected. This thickness can be selected by one skilled in the art according to the desired spatial resolution for each application.

In some embodiments, the conductive adhesive interface layer is a hydrogel and has an electrical conductivity of about 0.5~10 S/m, preferably, 2 S/m.

In some embodiments, the conductive adhesive interface layer is a hydrogel and has a Young's modulus of about 60~150 kPa, preferably, 80~130 kPa, more preferably 109.4 kPa.

In some embodiments, the conductive adhesive interface layer is a hydrogel and has an adhesion strength to moist biological tissue of about 20~30 kPa, preferably 25 kPa.

In some embodiments, the conductive adhesive interface layer is a hydrogel and has a peel strength to moist biological tissue of 20~35 Pa, preferably 28 Pa.

In some embodiments of the invention, the conductive adhesive interface layer is a hydrogel and has an adhesion strength to the insulating encapsulation layer of 100~150 kPa, preferably 124 kPa.

In some embodiments, the conductive adhesive interface layer is a hydrogel and has an average light transmittance in the visible light range greater than 85%, wherein the wavelength of visible light is 400-800 nm.

In some embodiments, after swelling in water for 60 minutes, the mass swelling ratio of the conductive adhesive interface layer is 86±11%.

In some embodiments of the invention, after swelling in water for 60 minutes, the volume swelling ratio of the conductive adhesive interface layer is 133±17%.

In some embodiments, the flexible insulating substrate comprises at least one of glass, silicon, polyimide, polycarbonate, perylene, polypropylene, polymethyl methacrylate (PMMA), polyethylene terephthalate (PETE), polydimethylsiloxane (PDMS), polyurethane, styrene-ethylene-butylene-styrene (SEBS), and combinations thereof.

In some embodiments, the insulating encapsulation layer comprises at least one of glass, silicon, polyimide, polycarbonate, perylene, polypropylene, polymethyl methacrylate (PMMA), polyethylene terephthalate (PETE), polydimethylsiloxane (PDMS), polyurethane, styrene-ethylene-butylene-styrene (SEBS), and combinations thereof.

In some embodiments, the thickness of the flexible insulating substrate is selected from 1 to 100 micrometers, such as 1, 1.5, 2, 2.5, 3, 3.5, 4, 4.5, 5, 6, 7, 8, 9, 10, 15, 20, 25, 30, 35, 40, 45, 50, 55, 60, 65, 70, 75, 80, 85, 90, 95, and 100 micrometers, and preferably 3 micrometers.

In some embodiments, the thickness of the insulating encapsulation layer is selected from 1 to 100 micrometers, such as 1, 1.5, 2, 2.5, 3, 3.5, 4, 4.5, 5, 6, 7, 8, 9, 10, 15, 20, 25, 30, 35, 40, 45, 50, 55, 60, 65, 70, 75, 80, 85, 90, 95, and 100 micrometers, and preferably 3 micrometers.

In some embodiments, the conductive circuit layer comprises one electrode and the insulating encapsulation layer comprises one opening. In other embodiments of the invention, the conductive circuit layer comprises a plurality of electrodes and the insulating encapsulation layer comprises a plurality of openings, wherein each of the plurality of electrodes independently corresponds to a respective one of the plurality of openings.

In some embodiments, the device further comprises a conductive polymer layer disposed between the electrodes and the filling portion. Preferably, the conductive polymer layer comprises a material selected from the group consisting of poly(3,4-ethylenedioxythiophene) (PEDOT), poly(3,4-ethylenedioxythiophene) polystyrene sulfonate (PEDOT:PSS), polythiophene, poly(p-phenylene sulfide), polypyrrole (PPy), polycarbazole, polyindole, polyazepine, polyaniline (PAni), and combinations thereof.

In some embodiments, the conductive circuit layer comprises: a first metal layer layered on a surface of the flexible insulating substrate; and a second metal layer layered on a side of the first metal layer away from the flexible insulating substrate, wherein the first metal layer comprises a metal different from that comprised in the second metal layer, and wherein the electrode is formed on or is part of the second metal layer. In some embodiments, the first metal layer comprises titanium, and/or the second metal layer comprises gold. In some embodiments, the first metal layer is made of titanium, and/or the second metal layer is made of gold.

In some embodiments, the hydrophilic polymer comprised in the conductive adhesive interface layer has a plurality of hydrophilic groups and is configured such that, when the conductive adhesive interface layer is put in contact with a moist biological tissue, the hydrophilic groups form multiple hydrogen bonds with nitrogen-containing or oxygen-containing functional groups of the biological tissue.

In some embodiments, in the conductive adhesive interface layer, a network formed by the hydrophilic polymer and a network formed by the hydrophobic polymer constitute an interpenetrating double-network structure, wherein the network formed by the hydrophobic polymer serves as a skeleton capable of restricting swelling of the network formed by the hydrophilic polymer. Meanwhile, in-situ polymerization can result in formation of topological entanglement at a molecular chain level between the conductive adhesive interface layer and the insulating encapsulation layer.

In some embodiments, the implantable bioelectronic device is a subdural μECoG device, especially for long-term implantation.

A second illustrative aspect of the invention provides a method for preparing an implantable bioelectronic device, comprising: providing a semi-encapsulated substrate, wherein the semi-encapsulated substrate comprises: a flexible insulating substrate; a conductive circuit layer disposed on the flexible insulating substrate and comprising an array of electrodes comprising one or more electrodes; and an insulating encapsulation layer encapsulating the conductive circuit layer and comprising an opening, wherein the electrodes are exposed through the opening; and forming a conductive adhesive interface layer on the insulating encapsulation layer, wherein the conductive adhesive interface layer comprises an exposed surface and a filling portion that extends into the opening, wherein the exposed surface is electrically connected to the electrodes through the filling portion, and wherein the conductive adhesive interface layer comprises a hydrophilic polymer and a hydrophobic polymer, and wherein the hydrophobic polymer comprises a polymerized network of silane-based monomers.

In some embodiments, the step of forming the conductive adhesive interface layer comprises: providing a solution comprising the hydrophilic polymer or monomers thereof in an organic solvent; mixing the solution with an initiator and the silane-based monomers to form a pre-polymerization solution; adding, such as dripping the pre-polymerization solution onto a surface of the insulating encapsulation layer and into the opening(s); and subjecting the initiator to initiation conditions to form the conductive adhesive interface layer from the pre-polymerization solution.

In some embodiments, the silane-based monomers each independently has a general formula: Rₐ(CH₂)ₙSi(OR_{b})₃, wherein Rₐ represents a group comprising a polymerizable functional group selected from the group consisting of an addition-polymerizable functional group, a condensation-polymerizable functional group, and a ring-opening polymerizable functional group; R_{b}s are the same or different, and are each independently selected from the group consisting of C₁₋₁₂ alkyl, and —C₁₂ alkylene-O—C₁₋₁₂ alkyl; and n is an integer of 0 to 12, preferably, n is an integer of 1 to 8, more preferably, n is an integer of 1 to 3.

In some embodiments, the silane-based monomers comprise: 3-(trimethoxysilyl)propyl methacrylate (TPM), vinyltrimethoxysilane (KH171), vinyltris (β-methoxyethoxy) silane (KH172), vinyltriethoxysilane (KH151), glycidoxypropyltrimethoxysilane, or combinations thereof. Preferably, the silane-based monomers are 3-(trimethoxysilyl)propyl methacrylate.

In some embodiments, the addition-polymerizable functional group is a carbon-carbon double bond or a carbon-carbon triple bond.

In some embodiments, the condensation-polymerizable functional group is an amino group, a carboxyl group, or a hydroxyl group.

In some embodiments, the ring-opening polymerizable functional group is a 3-20 membered epoxy group, a 4-20 membered lactone group, or a 4-20 membered lactam group.

In some embodiments, Ra is selected from at least one of vinyl, ethynyl, amino, methacryloyloxy, hydroxy, and glycidyl ether. Preferably, Ra is methacryloyloxy or glycidyl ether.

In some embodiments, R_{b}s are the same, and are each independently selected from the group consisting of C₁₋₆ alkyl, and —C₁₋₆ alkylene-O—C₁₋₆ alkyl.

In some embodiments, the hydrophilic polymer has a plurality of hydrophilic groups and is configured such that, when the conductive adhesive interface layer is put in contact with a moist biological tissue, the hydrophilic groups form multiple hydrogen bonds with nitrogen-containing or oxygen-containing functional groups of the biological tissue.

In some embodiments, the hydrophilic groups comprise one or more hydroxyl groups, one or more carboxyl groups, one or more amino groups, and/or one or more sulfonic acid groups.

In some embodiments, the hydrophilic polymer is selected from the group consisting of: polyvinyl alcohol (PVA), polyethylene oxide, hydrophilic polyurethane, hydrophilic epoxy resin, polyacrylamide, polyethylene glycol, poly(hydroxyethyl methacrylate), poly(hydroxyethyl acrylate), polyacrylic acid, copolymers thereof, and combinations thereof.

In some embodiments, wherein the number average molecular weight of the hydrophilic polymer is 1000 to 500000, preferably, is 5000 to 100000, such as 5000, 50000, 100000.

In some embodiments, the conductive adhesive interface layer is formed by in-situ polymerization of a composition comprising the hydrophilic polymer or monomers thereof and the silane-based monomers, particularly within the opening. Preferably, the pre-polymerization solution comprises 2-15 parts by weight of the hydrophilic polymer or monomers thereof, 0.1-10 parts by weight of the silane-based monomers, and 0.0001-0.5 parts by weight of an initiator. More preferably, in parts by weight, the pre-polymerization solution comprises: hydrophilic polymer 3-10 parts, silane-based monomer 0.15-4 parts, initiator 0.015-0.04 parts, organic solvent 30-60 parts.

In some embodiments, the mass ratio of the hydrophilic polymer to the hydrophobic polymer ranges from 3.5:1 to 17:1, including specific ratios such as about 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, 15:1, 16.5:1, or 17:1, preferably, the mass ratio is ranging from 4:1 to 12:1, more preferably, the mass ratio is about 7:1.

In some embodiments, the organic solvent is selected from the group consisting of: dimethyl sulfoxide, N,N-dimethylformamide, chloroform, tetrahydrofuran, toluene, ethanol, and ethylene glycol, and combinations thereof.

In some embodiments, the initiator is an oil-soluble thermal initiator or a photoinitiator.

In some embodiments, the oil-soluble thermal initiator is selected from the group consisting of: azobisisobutyronitrile, benzoyl peroxide, and combinations thereof.

In some embodiments, the photoinitiator is selected from the group consisting of: 2,2-dimethoxy-2-phenylacetophenone (DMPA), 2-hydroxy-2-methyl-1-phenyl-1-propanone, 1-hydroxycyclohexyl phenyl ketone, 2-methyl-1-[4-(methylthio)phenyl]-2-morpholinopropan-1-one, phenylbis(2,4,6-trimethylbenzoyl)phosphine oxide, 1-hydroxycyclohexyl phenyl ketone, 2-hydroxy-methylphenylpropan-1-one, benzoin dimethyl ether, and combinations thereof.

In some embodiments, the step of subjecting the initiator to initiation conditions comprises: irradiating the pre-polymerization solution with ultraviolet light having a wavelength of 365 nm and an intensity of 0.8 W/cm² for 10 to 50 minutes.

In some embodiments, the method further comprises a solvent exchange step in an aqueous solution to eliminate the organic solvent in the conductive adhesive interface layer. Preferably, the aqueous solution is ultrapure deionized water, and/or a buffered solution, such as a PBS solution.

In some embodiments, the method further comprises a dehydration step to dry the conductive adhesive interface layer.

A third illustrative aspect of the invention provides a human-machine interface system, comprising the implantable bioelectronic device according to the first aspect of the invention. Preferably, the system is a brain-computer interface system.

A fourth illustrative aspect of the invention provides use of a silane-based monomer having a general formula: Rₐ(CH₂)ₙSi(OR_{b})₃ for preparing a conductive adhesive interface layer on an implantable electrode, wherein the conductive adhesive interface layer is capable of: reducing neuroinflammation; reducing fibrotic tissue growth at the electrode-tissue interface; preserving electrical signal fidelity; and/or maintaining the electrode in position, and wherien Rₐ represents a group comprising a polymerizable functional group selected from the group consisting of an addition-polymerizable functional group, a condensation-polymerizable functional group, and a ring-opening polymerizable functional group; R_{b}s are the same or different, and are each independently selected from the group consisting of C₁₋₁₂ alkyl, and —C₁₋₁₂ alkylene-O—C₁₋₁₂ alkyl; and n is an integer of 0 to 12.

In some embodiments, the conductive adhesive interface layer comprises a hydrophilic polymer and a hydrophobic polymer formed by polymerization of a plurality of the silane-based monomers. Preferably, the silane-based monomer is 3-(trimethoxysilyl)propyl methacrylate.

In some embodiments, the hydrophilic polymer is selected from the group consisting of: polyvinyl alcohol (PVA), polyethylene oxide, hydrophilic polyurethane, hydrophilic epoxy, polyacrylamide, polyethylene glycol, polyhydroxy ethyl methacrylate, polyhydroxy ethyl acrylate, poly acrylic acid, copolymers thereof, and combinations thereof.

In some embodiments, the mass ratio of the hydrophilic polymer to the hydrophobic polymer ranges from 3.5:1 to 17:1, including specific ratios such as about 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, 15:1, 16.5:1, or 17:1, preferably, the mass ratio is ranging from 4:1 to 12:1, more preferably, the mass ratio is about 7:1.

In some embodiments, the implantable electrode is for providing an electric field, or an electrical stimulation to or detecting a biological signal of a subject carrying the implantable electrode, preferably, the biological signal comprises an electrocorti cogram (ECoG) signal, an electroencephalogram (EEG) signal, an electromyogram (EMG) signal, a cardiac electrogram signal, or an evoked compound action potential (ECAP) signal.

A fifth illustrative aspect of the invention provides a method for diagnosing, treating or preventing a disease in a subject in need thereof, comprising implanting the implantable bioelectronic device according to the first aspect of the invention into a tissue of the subject. Preferably, the implantable bioelectronic device comprises dehydrated conductive adhesive interface layer.

In some embodiments, the tissue is selected from the group consisting of brain, muscle and heart.

In some embodiments, the disease is selected from the group consisting of epilepsy, sleep and mental disorders (e.g. obsessive-compulsive disorder, deep depression, Parkinson's Disease), stroke, brain, spinal and peripheral nerve injuries, and neurodegenerative diseases (e.g. ALS, other forms of locked⁻¹n syndrome).

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention can be more fully understood by reading the following detailed description together with the accompanying drawing, in which like reference indicators are used to designate like elements, and in which:

FIG. 1 schematically shows an intracranial installation of an implantable bioelectronic device according to an embodiment of the invention.

FIG. 2 shows an exploded structural view of an implantable bioelectronic device according to an embodiment of the invention.

FIG. 3 shows a flowchart of a method for preparing an implantable bioelectronic device according to an embodiment of the invention.

FIG. 4 shows a structural block diagram of a human-machine interface system according to an embodiment of the invention.

FIG. 5 schematically illustrates the aGel synthesis process.

FIG. 6 shows FT-IR spectra of the PVA/PTPM, PVA/TPM, and pure PVA solutions.

FIG. 7 shows comparison of properties of PVA/PTPM hydrogels with different compositions. a) Ionic conductivity. b) Swelling kinetics of hydrogels upon immersion in 1×PBS. c) Tissue adhesion strength measured by lap-shear tests. d) Average optical transmittance of hydrogel coatings on glass slides as a function of mass ratio and coating thickness. Data are presented as mean±SD.

FIG. 8 shows quantitative analysis of protein adsorption on the surfaces of PI, aGel, and PVA (n=3). Data are presented as mean±SEM. Data following a normal distribution were analyzed using one-way ANOVA; otherwise, the Kruskal-Wallis test was used for significance analysis. *p<0.05, **p<0.01, ***p<0.001.

FIG. 9 shows quantitative analysis of NIH3T3 cell density on control (glass), PI, and aGel-PI substrates after 1, 3, and 5 days of culture (n=3). Data are presented as mean±SEM. Data following a normal distribution were analyzed using one-way ANOVA; otherwise, the Kruskal-Wallis test was used for significance analysis. *p<0.05, **p<0.01, ***p<0.001.

FIG. 10 shows cryo-scanning electron microscopy characterization of aGel morphology.

FIG. 11 shows normalized iso-potential map showing the effects of varying aGel thickness t (0-100 μm) and aGel conductivity σ (0.01-10 S m⁻¹). The potential (V) was normalized to the maximum potential obtained at the geometric origin d₀ under aGel-free conditions. The white star indicates the optimal design parameter chosen to achieve high electrical fidelity (t=~10 μm, σ=~2 S m⁻¹).

FIG. 12 is a representative image of the 1024-channel aGel-μECoG array.

FIG. 13 shows impedance at 1 kHz for devices with as-fabricated bare Au electrodes, ECoG (electroplated with PEDOT:PSS), and aGel-μECoG with coating thicknesses of 10 and 100 m. Data were collected from more than 60 electrodes across three independent samples and are presented as the mean±SD.

FIG. 14 shows normalized SSEP potentials across varying stimulation intensities (50, 100, 200, 500, and 1000 μA) for three device types (sample sizes: μECoG, n=7, 6, 8, 7, 8; 10-μm aGel-μECoG, n=7, 7, 8, 8, 8; 100 μm aGel-μECoG, n=7, 8, 8, 8, 8). Statistical analysis was performed using an unpaired t-test for a specific stimulation condition and two-way ANOVA for comparisons among device types under all stimulation conditions.

FIG. 15 shows bandpass-filtered spatial maps of signal-to-noise ratio (SNR) at a stimulation intensity of 200 μA.

FIG. 16 shows estimation of spatial resolution using the fitted parameter λ across frequency bands (n=7 per group; exponential decay fit: y=a·exp(−xλk)+c). Data are presented as mean±SEM, unless otherwise specified. ns, not significant; *p<0.05, **p<0.01, ***p<0.001.

FIG. 17A-I shows histological evaluation of the in vivo biocompatibility of −aGel and +aGel. Data are presented as mean±SEM.

FIG. 18 shows quantitative analysis of fibrotic tissue thickness in brain tissue two weeks post-implantation with +aGel or −aGel (n=18 from three independent rats). Data are presented as mean±SEM.

FIG. 19 are representative photos of −aGel (left) and +aGel (right) films retrieved 8 weeks after in vivo implantation.

FIG. 20 shows distinct 1 kHz impedance trends of μECoG and aGel-μECoG over 16 weeks (n=5 rats).

FIG. 21 shows equivalent circuit models for in vitro and in vivo conditions.

FIGS. 22A and 22B show EIS and fitted curves for μECoG (FIG. 22A) and aGel-ECoG (FIG. 22B) on 0, 12, 30, and 60 days post-implantation.

FIG. 23 shows changes in the Z_{CPE} exponent term a and capacitance at the electrode-tissue interface for μECoG and aGel-μECoG over a 60-day implantation period. n=6 from 2 rats on each testing day. Data are presented as mean±SEM.

FIG. 24 shows changes in the resistive and capacitive components (R_{T} and C_{T}) of the surrounding tissue for μECoG and aGel-μECoG over the 60-day implantation period. n=6 from 2 rats on each testing day. Data are presented as mean±SEM.

FIG. 25 shows SNR of 8 Hz harmonics for aGel-μECoG and μECoG electrodes over 16 weeks. N>200 independent electrodes from more than 4 rats for both types of arrays across all time points. The central line indicates the median, the box represents the interquartile range (IQR), the whiskers denote the SD, and the white dots represent mean values.

FIG. 26 shows LMM-fitted SNR over weeks post-implantation.

FIG. 27 shows coefficient of variation of the 8-Hz SNR shown in FIG. 25. The box represents the interquartile range (IQR), the whiskers denote the SD, and the white dots represent mean values.

FIG. 28 shows quantitative statistical analysis of fibrotic tissue thickness on the brain surface and beneath the device after 16 weeks of implantation with μECoG and aGel-μECoG electrodes (n=18 from three independent rats). Data are presented as mean±SEM.

### DETAILED DESCRIPTION OF THE INVENTION

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “an electrode” includes a plurality of such electrodes and reference to “the measurement” includes reference to one or more measurements and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about,” particularly in reference to a given quantity, is meant to encompass deviations of plus or minus 20%, for example, 15%, 10%, or 5% or less.

The terms “individual”, “subject” and “patient”, are used interchangeably herein and refer to any subject with a brain, including invertebrates and vertebrates such as, but not limited to, arthropods (e.g., insects, crustaceans, arachnids), cephalopods (e.g., octopuses, squids), amphibians (e.g., frogs, salamanders, caecilians), fish, reptiles (e.g., turtles, crocodilians, snakes, amphisbaenians, lizards, tuatara), mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

In a first aspect of the present invention, provided is a device that features a conductive adhesive interface layer that serves as an interface between the device and the surrounding tissue (such as the brain of a subject) once implanted in position. The conductive adhesive interface layer can provide molecular-level topological entanglement and form a physically interlocked structure with the surface to which it attaches to, such as a surface of the insulating encapsulation layer, or a surface of the electrode. This enhances the bonding strength between the conductive adhesive interface layer and the substrate, preventing delamination. The conductive adhesive interface layer is composed of hydrophilic and hydrophobic polymer networks and is in a dehydrated form. After being implanted, it can rapidly absorb interfacial water from the surrounding tissue surface, forming a hydrogel and promoting close contact with the tissue. Strong and tough wet adhesion to the brain tissue is achieved through non-covalent interactions, such as multiple hydrogen bonds with the hydrophilic network, thereby inhibiting fretting damage. The hydrogel exhibits enhanced mechanical strength, toughness, fatigue resistance, and anti-swelling performance, capable of withstanding complex and dynamic in vivo environments and maintaining functional reliability.

It should be noted that the implantable bioelectronic device according to embodiments of the invention can be implanted not only intracranially (especially subdural) but also in the torso (e.g., the heart) and limbs (e.g., arms, legs).

FIG. 1 schematically shows an intracranial installation position diagram of an implantable bioelectronic device according to an embodiment of the invention.

As shown in FIG. 1, the dura mater 14 is located between the skull 12 and the brain 16, forming an important protective membrane layer within the cranium. The implantable bioelectronic device 100 can be attached to the inner side of the dura mater 14 (i.e., the side facing the cerebral cortex) via subdural implantation, enabling direct monitoring of the electrical activity of the cerebral cortex. For example, the implantable bioelectronic device 100 can cover the motor cortex, sensory cortex, or language-related functional areas for acquiring (especially long-term, continuous) neural signals such as local field potentials (LFPs), high-frequency oscillations (HFOs), and event-related potentials (ERPs). The device 100 is suitable for clinical and research scenarios such as epileptogenic zone localization, brain function mapping, and brain-computer interfaces.

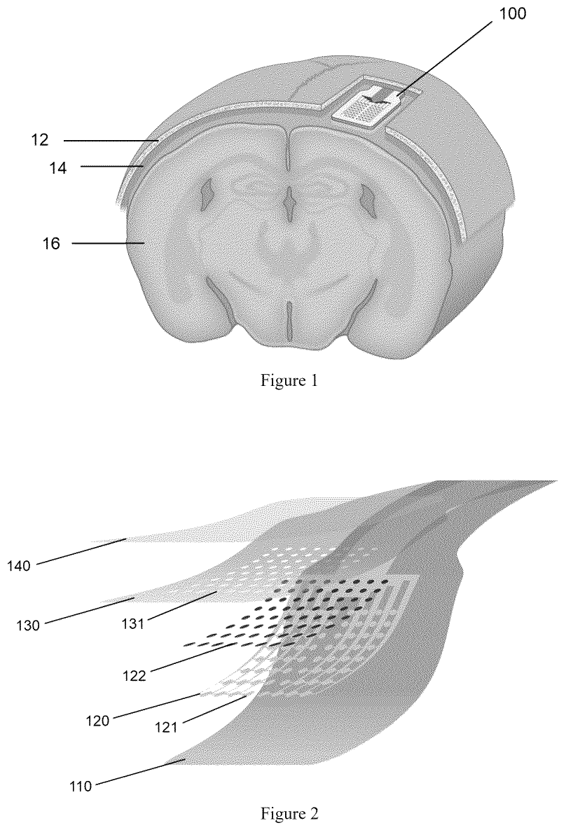

FIG. 2 schematically shows an exploded structural view of an implantable bioelectronic device according to an embodiment of the invention.

As shown in FIGS. 1 and 2, the implantable bioelectronic device 100 may comprise a flexible insulating substrate 110, a conductive circuit layer 120, an insulating encapsulation layer 130, and a conductive adhesive interface layer 140. To facilitate viewing and understanding the relative positions and structures of each layer, FIG. 2 shows the implantable bioelectronic device 100 in an exploded view.

The flexible insulating substrate 110 serves as support of the device 100, providing both mechanical support and electrical insulation for the conductive circuit layer 120.

The flexible insulating substrate 100 can be fabricated of materials selected from glass, silicon, polyimide (PI), polycarbonate, perylene, polypropylene, polymethyl methacrylate (PMMA), polyethylene terephthalate (PETE), polydimethylsiloxane (PDMS), polyurethane, styrene-ethylene-butylene-styrene (SEBS), and combinations thereof. Preferably, the flexible insulating substrate 100 is fabricated of PI which has excellent flexibility and biocompatibility.

When the implantable bioelectronic device 100 is used intracranially, the flexible insulating substrate 110 should be flexible enough to conform to the curvature of the brain, causing no stress concentration under minor deformations or pulsations of brain tissue, so as to avoid damaging the brain tissue.

In some embodiments, the thickness of the flexible insulating substrate 110 is selected from 1 μm to 100 μm, for example, 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, and any value therebetween.

In some embodiments, the flexible insulating substrate 110 can have a regular or irregular geometric shape. Exemplarily, the flexible insulating substrate 110 can be rectangular. The length of the flexible insulating substrate 110 is selected from 1-100 mm, and the width of the flexible insulating substrate 110 is selected from 1-100 mm.

As shown in FIG. 2, the conductive circuit layer 120 is disposed on the flexible insulating substrate 110 and constitutes the conductive pathway for reading neuroelectrophysiological signals. The conductive circuit layer 120 comprises an array of electrodes comprising one or more electrodes 121. During use, the electrode(s) 121 can be connected to an external device (e.g., a signal receiving device, or a signal generating device) via wired or wireless means. The electrode(s) 121 can communicate with external devices. For example, the electrode(s) 121 can acquire electrical signals from the implantation area and transmit them to the external device. Alternatively, the external device can transmit stimulation signals to the electrode(s) 121, which then apply the stimulation signals to the implantation area.

When the implantable bioelectronic device 100 is used intracranially, the electrode(s) 121 are generally used to acquire weak electrical signals generated by the cerebral cortex and transmit these signals to an external device. The external device may, for example, perform further analysis on the signals after signal processing such as amplification.

Exemplarily, when the conductive circuit layer 120 contains a plurality of electrodes 121, the electrodes 121 can be arranged in an array, such as an 8×8, 16×16, or other rectangular arrays. Furthermore, circumferential arrays are also feasible.

Exemplarily, the diameter of an electrode(s) 121 can be 10 mm to 30 mm, for example, 20 mm. The spacing between two adjacent electrodes 121 can be 100 micrometers to 300 mm, for example, 200 mm.

The conductive circuit layer 120 may comprise one or more metal materials.

In some embodiments, the conductive circuit layer 120 comprises only one metal material. For example, the conductive circuit layer 120 may comprise gold.

In other embodiments, the conductive circuit layer 120 may comprise a first metal layer (not shown) and a second metal layer (not shown) sequentially stacked. The first metal layer is stacked on the surface of the flexible insulating substrate 110, and the second metal layer is stacked on the side of the first metal layer away from the flexible insulating substrate 110. The material of the first metal layer is different from the material of the second metal layer. The electrode(s) 121 is formed on or is part of the second metal layer. The first metal layer can serve as an adhesion layer between the second metal layer and the flexible insulating substrate 110 to improve the adhesion of the conductive circuit layer 120 to the flexible insulating substrate 110. The second metal layer can serve as the functional layer for electrical signal transmission.

The thickness of the first metal layer can be 10 nm to 300 nm, for example, 10, 20, 30, 40, 50, 60, 70, 80, 100, 110, 120, 130, 140, 150, 160, 170, 180, 200, 210, 220, 230, 240, 250, 260, 270, 280, or 300 nm.

Exemplarily, the first metal layer can be or comprise titanium (Ti), and the second metal layer can be or comprise gold (Au). Titanium has high chemical activity and can form stable chemical bonds or a diffusion interface with the flexible insulating substrate 110 (e.g., polyimide), significantly enhancing the adhesion between the metal material and the organic substrate. Gold has excellent conductivity, chemical inertness, and biocompatibility, is not easily oxidized or corroded in body fluid environments, and can stably transmit weak neural signals over a long term. Furthermore, the gold surface is amenable to subsequent functionalization treatments (e.g., modification with conductive polymers) to further reduce interface impedance and improve signal quality.

As shown in FIG. 2, in some embodiments, a conductive polymer layer 122 can be formed on the surface of the electrode(s) 121. The conductive polymer layer 122 can comprise a material selected from poly(3,4-ethylenedioxythiophene) (PEDOT), poly(3,4-ethylenedioxythiophene) polystyrene sulfonate (PEDOT:PSS), polythiophene, poly(p-phenylene sulfide), polypyrrole (PPy), polycarbazole, polyindole, polyazepine, polyaniline (PAni), and combinations thereof.

Exemplarily, the conductive polymer layer 122 can comprise poly(3,4-ethylenedioxythiophene)-polystyrene sulfonate (PEDOT:PSS) to reduce electrochemical impedance. Thus, the conductive circuit layer 120 can be a PEDOT:PSS/gold nanocomposite conductive layer, which can be formed, for example, via an in-situ electrodeposition process. The formation method will be further explained below in the preparation processsection.

As shown in FIG. 2, the insulating encapsulation layer 130 encapsulates the conductive circuit layer 120, such that the conductive circuit layer 120 is encapsulated by the flexible insulating substrate 110 and the insulating encapsulation layer 130. Furthermore, the insulating encapsulation layer 130 comprises opening(s) 131. Typically, the number of openings 131 matches the number of electrodes 121. For example, when the number of electrode 121 is one, the number of opening 131 is one, and vice versa. When the number of electrodes 121 is a plurality, the number of openings 131 is a plurality, and each of the plurality of electrodes 121 independently corresponds to a respective one of the plurality of openings 131. Different electrodes 121 are isolated from each other by the insulating encapsulation layer, thereby providing electrical isolation. This layer serves to cover all metal traces but the electrodes, thereby preventing signal crosstalk and body fluid penetration, while also protecting the metal material of the conductive circuit layer from corrosion by body fluids, enhancing the long-term stability and reliability of the device.

In some embodiments, the insulating encapsulation layer 130 can comprise or be fabricated of materials selected from glass, silicon, polyimide, polycarbonate, perylene, polypropylene, polymethyl methacrylate (PMMA), polyethylene terephthalate (PETE), polydimethylsiloxane (PDMS), polyurethane, styrene-ethylene-butylene-styrene (SEBS), and combinations thereof.

Exemplarily, the insulating encapsulation layer 130 can comprise or be made of the same material as the flexible insulating substrate 110. For example, the insulating encapsulation layer 130 can be made of a flexible polymer material such as PI.

In some embodiments, the thickness of the insulating encapsulation layer 130 is selected from 1 μm to 100 μm, for example, 1 μm, 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, and any value therebetween. Exemplarily, the thickness of the insulating encapsulation layer 130 is the same as the thickness of the flexible insulating substrate 110. Exemplarily, the thickness of the insulating encapsulation layer 130 is larger than the thickness of the flexible insulating substrate 110. Exemplarily, the thickness of the insulating encapsulation layer 130 is smaller than the thickness of the flexible insulating substrate 110.

In some embodiments, the insulating encapsulation layer 130 can have a regular or irregular geometric shape. Exemplarily, the insulating encapsulation layer 130 can be rectangular. The length of the insulating encapsulation layer 130 is selected from 1-100 mm, and the width of the insulating encapsulation layer 130 is selected from 1-100 mm. Exemplarily, the shape of the insulating encapsulation layer 130 is the same as the shape of the flexible insulating substrate 110, and the size of the insulating encapsulation layer 130 is the same as the size of the flexible insulating substrate 110.

The shape of the opening 131 depends on the shape of the electrode 121, and typically their shapes are consistent. The shape of the opening 131 or the electrode 121 can be, for example, circular, square, or any other shape, which is not limited in the embodiments of the invention.

As shown in FIG. 2, the conductive adhesive interface layer 140 comprises an exposed surface and a filling portion that extends into the opening 131. The exposed surface is the surface of the conductive adhesive interface layer 140 away from the flexible insulating substrate 110. During use, this exposed surface contacts the biological tissue surface (e.g., the brain tissue surface). The filling portion is typically located inside the opening 131 and is in contact with the electrode 121. The conductive adhesive interface layer 140 is electrically conductive; therefore, the exposed surface is electrically connected to the electrode 121 through the filling portion. In embodiments with a conductive polymer layer 122, the conductive polymer layer 122 is located between the filling portion and the electrode 121.

The conductive adhesive interface layer 140 comprises a hydrophilic polymer and a hydrophobic polymer. The hydrophobic polymer comprises a polymerized network of silane-based monomers. The conductive adhesive interface layer 140 is formed by in-situ polymerization of the hydrophilic polymer and the hydrophobic polymer within the opening. The network formed by the hydrophilic polymer and the network formed by the hydrophobic polymer constitute an interpenetrating double-network structure, wherein the network formed by the hydrophobic polymer serves as a skeleton, restricting the swelling of the network formed by the hydrophilic polymer.

The hydrophilic polymer has multiple hydrophilic groups and is configured such that, when the conductive adhesive interface layer is connected to moist biological tissue, the hydrophilic groups form multiple hydrogen bonds with nitrogen-containing or oxygen-containing functional groups of the moist biological tissue, enhancing interface affinity. The hydrophilic groups can be selected from hydroxyl groups, carboxyl groups, or sulfonic acid groups.

The hydrophilic polymer is selected from the group consisting of: polyvinyl alcohol (PVA), hydrophilic polyurethane, hydrophilic epoxy, polyacrylamide, polyethylene glycol, polyhydroxy ethyl methacrylate, polyhydroxy ethyl acrylate, poly acrylic acid, copolymers thereof, and combinations thereof.

The conductive adhesive interface layer 140 has a hydrophilic/hydrophobic interpenetrating double-network structure, enabling rapid water absorption, swelling, and formation of close contact with the brain tissue surface. The interpenetrating double-network structure is formed on the surface of the insulating encapsulation layer 130 via in-situ polymerization. Through this polymerization method, molecular-level topological entanglement is formed between the interpenetrating double-network structure and the insulating encapsulation layer 130, thereby enhancing the interface bonding strength. Specifically, this in-situ polymerization method allows the interpenetrating double-network structure to form a physical interlock with the surface of the insulating encapsulation layer 130, ensuring a strong, low-impedance, and biocompatible stable connection between the hydrogel and the underlying electronic substrate, thereby guaranteeing structural integrity and efficient charge transfer during long-term implantation.

Specifically, upon implantation of the implantable bioelectronic device 100, the dry conductive adhesive interface layer 140 instantaneously absorbs interfacial water from the brain tissue surface and achieves immediate, strong, tough, and reversible wet adhesion to the brain tissue surface through a network of non-covalent interactions (e.g., multiple hydrogen bonds), effectively inhibiting interface friction damage caused by physiological micromotions.

In some embodiments, the hydrophobic polymer is formed by polymerization of 3-(trimethoxysilyl)propyl methacrylate (PTPM) monomers. The hydrophobic polymer network can be formed by in-situ polymerization of 3-(trimethoxysilyl)propyl methacrylate (PTPM) monomers into a cross-linked network, serving as the mechanical skeleton of the interpenetrating double network, effectively inhibiting excessive swelling of the hydrophilic network (e.g., PVA) in the hydrated state, and preventing significant volume change or deformation of the conductive adhesive interface layer, thereby maintaining structural integrity. Simultaneously, the trimethoxysilyl groups in PTPM can undergo condensation reactions with functional groups (e.g., —OH, —COOH) on the surface of the insulating encapsulation layer (e.g., polyimide PI) during polymerization, forming covalent bonds and achieving molecular-level topological entanglement. This significantly enhances the interfacial bonding strength between the conductive adhesive interface layer and the substrate, avoiding delamination or peeling caused by mechanical disturbance or swelling stress during long-term implantation. Thus, the conductive adhesive interface layer 140 combines high toughness with low modulus characteristics, and its Young's modulus highly matches that of brain tissue, significantly reducing interfacial stress concentration. Good interfacial mechanical matching can effectively inhibit immune cell activation and fibroblast migration, block the formation pathway of fibrotic encapsulation, minimize neuroinflammatory responses and fibrotic capsule thickness, thereby ensuring long-term stable operation of the electrode in vivo and enabling continuous, high-fidelity neural signal monitoring.

Exemplarily, the thickness of the conductive adhesive interface layer is 5~15 μm, such as 5, 6, 7, 8, 9, 10, 11, 12, 13, 14, 15 μm or any number therebetween. In some embodiments, the conductive adhesive interface layer has a thickness of 10 μm.

Exemplarily, the conductivity of the conductive adhesive interface layer is 0.5~10 S/m, preferably, 2 S/m.

Exemplarily, the Young's modulus of the conductive adhesive interface layer is 60~150 kPa, preferably, 80~130 kPa, more preferably 109.4 kPa.

Exemplarily, the adhesion strength of the conductive adhesive interface layer to moist biological tissue is 20~30 kPa, preferably 25 kPa.

the peel strength of the conductive adhesive interface layer from moist biological tissue is 20~35 Pa, preferably 28 Pa.

the adhesion strength of the conductive adhesive interface layer to the insulating encapsulation layer is 100~150 kPa, preferably 124 kPa.

Through mechanical performance tests, biotoxicity tests, and long-term implantation tests, chronic histological analysis and electrochemical impedance spectroscopy conducted on hydrogel-integrated electrode arrays implanted subdurally in rat models have shown that the implantable bioelectronic device provided by the invention significantly reduces neuroinflammation and fibrotic capsule formation at the electrode-tissue interface. In long-term implantation models, the implantable bioelectronic device provided by the invention exhibited significantly reduced intensity of interfacial inflammatory response, with fibrotic capsule thickness controlled at the micrometer level; electrochemical impedance maintained over 90% of the initial value across a wide frequency range, and the attenuation rate of the signal-to-noise ratio for neuroelectrophysiological signals (including SSVEP and LFP characteristic bands) improved by more than 2 times compared to the control group.

In a further aspect, the invention also provides a method for producing an implantable bioelectronic device.

The method comprises:
(1) providing a semi-encapsulated substrate; wherein the semi-encapsulated substrate comprises a flexible insulating substrate; a conductive circuit layer disposed on the flexible insulating substrate and comprising an array of electrodes comprising one or more electrodes; and an insulating encapsulation layer, encapsulating the conductive circuit layer and comprising an opening, wherein the electrode is exposed through the opening; and
(2) forming an conductive adhesive interface layer on the insulating encapsulation layer, wherein the conductive adhesive interface layer comprises an exposed surface and a filling portion that extends into the opening, the exposed surface is electrically connected to the electrode through the filling portion, and wherein the conductive adhesive interface layer comprises a hydrophilic polymer and a hydrophobic polymer, and wherein the hydrophobic polymer is formed comprises a polymerized network of silane-based monomers, such as 3-(trimethoxysilyl)propyl methacrylate (TPM) monomers.

The step of providing the semi-encapsulated substrate comprises: providing a flexible insulating substrate; forming a conductive circuit layer comprising an array of electrodes comprising one or more electrodes on the flexible insulating substrate; forming an insulating encapsulation layer on the conductive circuit layer, and forming corresponding one or more openings in regions of the insulating encapsulation layer encapsulating the one or more electrodes.

The step of forming the conductive adhesive interface layer comprises: providing a solution comprising the hydrophilic polymer or monomers thereof in an organic solvent; mixing the solution with the silane-based monomers and an initiator to form a pre-polymerization solution; adding the pre-polymerization solution onto a surface of the insulating encapsulation layer and into the opening(s); and subjecting the initiator to initiation conditions to form the conductive adhesive interface layer from the pre-polymerization solution.

Specifically, the step of forming the conductive adhesive interface layer comprises: adding the pre-polymerization solution onto a surface of the insulating encapsulation layer and into the opening(s). Through in-situ polymerization, the hydrophilic polymer or monomers thereof and the hydrophobic monomers are simultaneously polymerized on the surface of the insulating encapsulation layer to form the conductive adhesive interface layer with an interpenetrating double-network structure, and molecular-level topological entanglement is formed between the interpenetrating double-network structure and the insulating encapsulation layer.

The hydrophilic polymer is selected from: polyvinyl alcohol (PVA), hydrophilic polyurethane, hydrophilic epoxy, polyacrylamide, polyethylene glycol, polyhydroxy ethyl methacrylate, polyhydroxy ethyl acrylate, poly acrylic acid, copolymers thereof, and combinations thereof.

The photoinitiator is selected from 2,2-dimethoxy-2-phenylacetophenone (DMPA), 2-hydroxy-2-methyl-1-phenyl-1-propanone, 1-hydroxycyclohexyl phenyl ketone, 2-methyl-1-[4-(methylthio)phenyl]-2-morpholinopropan-1-one, phenylbis(2,4,6-trimethylbenzoyl)phosphine oxide, and combinations thereof.

The mass ratio of the hydrophilic polymer or monomers thereof to the 3-(trimethoxysilyl) propyl methacrylate monomers ranges from 3.5:1 to 17:1, including specific ratios such as about 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, 15:1, 16.5:1, or 17:1, preferably, the mass ratio ranges from 4:1 to 12:1, more preferably, the mass ratio is about 7:1.

In some embodiments, the implantable bioelectronic device 100 described above can be prepared using this preparation method. However, embodiments of the invention do not exclude other methods for preparing the implantable bioelectronic device 100 described above.

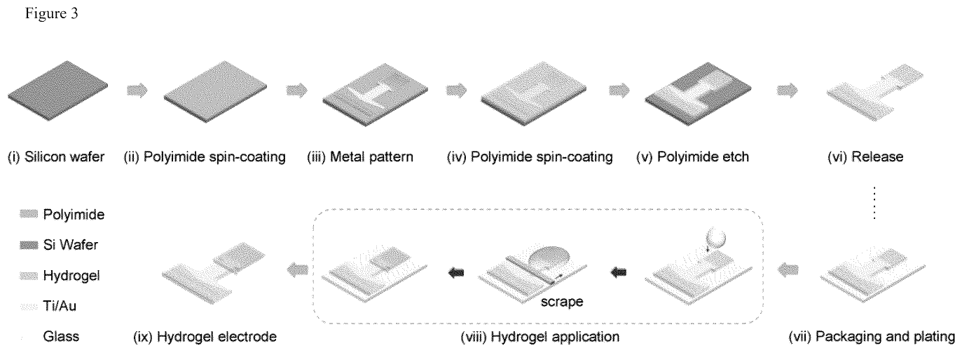

FIG. 3 schematically shows a flowchart of a method for preparing an implantable bioelectronic device according to an embodiment of the invention.

Please refer to both FIG. 2 and FIG. 3. As shown in subfigure (i) of FIG. 3, a temporary carrier substrate is provided. For example, the temporary carrier substrate can be a silicon (Si) wafer, with a size of, for example, 4×4 inches. The silicon wafer has a flat surface, good thermal stability, and good process compatibility with organic materials such as polyimide (PI), making it suitable as a support carrier in the fabrication process of flexible devices.

As shown in subfigure (ii) of FIG. 3, the flexible insulating substrate 110 is formed on the temporary carrier substrate. For example, a spin-coating technique can be used to uniformly apply a liquid PI precursor solution onto the surface of the silicon wafer. After gradient baking and imidization curing, a first PI film with a thickness of about 3 μm is formed, serving as the flexible insulating substrate 110. The flexible insulating substrate 110 can not only provide mechanical support but also possesses excellent electrical insulation, chemical stability, and biocompatibility, forming the foundation for the subsequent multilayer structure. Optionally, a sacrificial layer made of Ni, or parylene C can be formed on the temporary carrier substrate before forming the flexible insulating substrate 110.

As shown in subfigures (iii) of FIG. 3, the conductive circuit layer 120 and the electrodes 121 are formed on the flexible insulating substrate 110 via a physical vapor deposition (PVD) technique. For example, a magnetron sputtering method can be used to sequentially deposit a 50 nm thick titanium (Ti) layer and a 150 nm thick gold (Au) layer on the PI substrate. Here, Ti serves as an adhesion layer to enhance the bonding strength between Au and PI. Subsequently, standard photolithography is used to define the electrode and lead patterns, and wet etching or a lift-off process is employed to form the precise conductive trace structure, ultimately obtaining the conductive circuit layer 120 composed of Ti/Au bilayer metal and the multiple electrodes 121 distributed thereon.

As shown in subfigures (iv) of FIG. 3, the insulating encapsulation layer 130 is formed on the conductive circuit layer 120. For example, the spin-coating process can be used again to apply a layer of liquid polyimide over the entire substrate. After similar baking and curing, a second PI film with a thickness of about 3 μm is formed as the initial insulating encapsulation layer 130. This layer is used to cover all metal traces except the electrodes, preventing signal crosstalk, body fluid penetration, and metal corrosion, ensuring the long-term operational reliability of the device.

As shown in subfigures (v) of FIG. 3, the initial insulating encapsulation layer 130 is patterned in the regions encapsulating the respective electrodes 121 to form the corresponding openings 131. For example, photolithography combined with reactive ion etching can be used: first, spin-coating a photoresist on the PI surface, followed by exposure and development; then defining the opening areas and using oxygen plasma for dry etching to remove the PI material at the corresponding areas to form circular or square openings 131 with a diameter of about 100-200 m, thereby exposing the underlying electrodes 121. This ensures that the subsequent hydrogel (i.e., the conductive adhesive interface layer) only contacts the electrode areas of the conductive circuit layer, achieving precise electrical connection.

As shown in subfigure (vi) of FIG. 3, the device with the completed multilayer structure is released from the silicon carrier substrate. For example, this can be achieved by selectively etching a sacrificial layer between the PI substrate and silicon wafer, or by immersing the device on the wafer in deionized water and peeling off the device. A complete flexible film device can thus be easily obtained by utilizing the weak interface characteristics of PI itself.

As shown in subfigure (vii) of FIG. 3, the released flexible device is transferred to a temporary glass carrier substrate. Wire bonding (such as gold ball bonding) is then performed to form solder bumps at the lead ends, followed by electroplating to thicken the leads, improving their mechanical strength and conductivity, facilitating subsequent packaging and connection to external circuits.

As shown in subfigure (viii) of FIG. 3, on the glass substrate, the pre-polymerization solution containing the hydrophilic polymer or monomers thereof, the hydrophobic monomers, and the photoinitiator is dispensed into the area corresponding to each opening 131. The amount of the pre-polymerization solution is controlled at the nanoliter to microliter level to ensure it only fills the opening 131 areas without overflowing. Then, a micrometer-level scraper or a pressing mold process can be used to level the pre-polymerization solution in the opening 131 areas, forming a liquid film with uniform thickness and highly controllable height, avoiding bubbles or uneven thickness that could affect the polymerization quality. Then, ultraviolet (UV) light is applied to the areas where the pre-polymerization solution was dispensed to induce the in-situ polymerization reaction. Specifically, the electrode array coated with the hydrogel can be placed under UV light with a wavelength of 365 nm and an intensity of 0.8 W/cm² for 10 minutes to 50 minutes (especially 30 minutes) to promote full cross-linking and curing of the hydrogel network. Subsequently, the device is immersed in ultrapure deionized water for solvent exchange for 30 minutes to remove residual organic solvent. Then, the device is transferred to sterile phosphate-buffered saline (PBS) and soaked for 72 hours, during which the PBS is changed daily, to completely remove any residual soluble components such as dimethyl sulfoxide (DMSO).

As shown in subfigure (ix) of FIG. 3, the device is removed from the glass substrate, finalizing the preparation of the implantable bioelectronic device 100. The resulting electrode has the typical multilayer structure shown in FIG. 2: from bottom to top, the flexible insulating substrate 110, the conductive circuit layer 120 (including one or more electrodes 121), the insulating encapsulation layer 130 (with one or more openings 131), and the conductive adhesive interface layer 140. The overall thickness of this device is less than 20 μm, possessing excellent flexibility, biocompatibility, and long-term stability, suitable for subdural implantation for neural signal monitoring.

In a preferred embodiment, the device is baked for example at 60° C. for 15 minutes to dehydrate the conductive adhesive interface layer in the form of a hydrogel and bring it into a dry state for storage. The dehydrated device is then sealed in sterile packaging and subjected to UV irradiation for surface sterilization for 30 minutes to ensure the device is sterile, meeting the biosafety requirements for implantable medical devices.

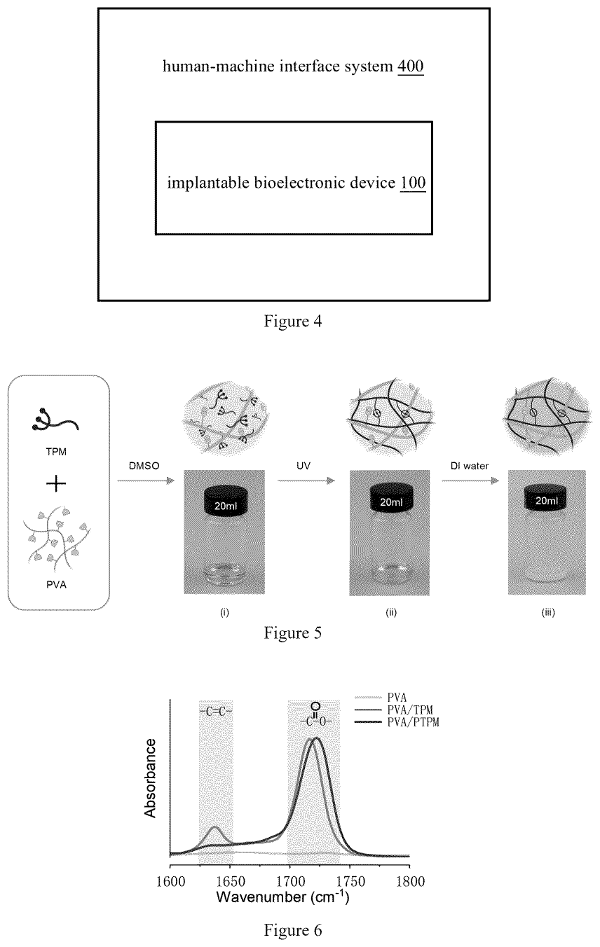

Based on the aforementioned implantable bioelectronic device 100, the invention also provides a human-machine interface system. FIG. 4 schematically shows a structural block diagram of a human-machine interface system according to an embodiment of the invention.

As shown in FIG. 4, the human-machine interface system 400 includes the implantable bioelectronic device(s) 100. The number of implantable bioelectronic devices 100 in the human-machine interface system 400 can be one or more, which is not limited by the invention. The implantable bioelectronic device 100 serves as the core sensing component, implanted on the subdural surface, and can be used for non-invasive, high spatiotemporal resolution acquisition of neuroelectrophysiological signals such as local field potentials (LFP), event-related potentials (ERP), or high-frequency oscillations (HFO) from the cerebral cortex.

Besides the implantable bioelectronic device(s) 100, the human-machine interface system 400 may also include other components. For example, a signal amplification and conditioning module, a data processing unit, a wireless communication module, a power management module, a feedback stimulation unit, a user interface, a storage module, and/or a security and encryption module, etc.

The signal amplification and conditioning module can be used to amplify and filter the weak electrical signals acquired from the electrodes to improve the signal-to-noise ratio and remove interference signals.

The data processing unit may, for example, include an analog-to-digital converter (ADC) and a digital signal processor (DSP), responsible for converting analog signals to digital signals and executing necessary signal processing algorithms (e.g., feature extraction, pattern recognition).

The wireless communication module can enable data transmission with external devices, using common technologies such as Bluetooth, Wi-Fi, or dedicated medical band radio, ensuring that data can be transmitted stably and in real-time to external computing devices or cloud servers.

The power management module can provide stable power support for the entire system, as well as power management integrated circuits, to optimize energy efficiency and extend operational time. The feedback stimulation unit, in certain applications (e.g., epilepsy treatment or motor function restoration), can apply electrical stimulation or other forms of feedback to the patient based on the parsed brain activity state. This unit is responsible for generating and delivering precisely controlled stimulation pulses.

For some semi-implantable or external components, a user interface facilitating operation and monitoring can be provided, such as a display screen, buttons, or a touch panel, or it can be implemented via a smartphone application for remote monitoring and parameter adjustment.

A storage module can be used to save raw data and processed results for subsequent analysis or as part of long-term health records.

A security and encryption module can ensure the security of transmitted data and prevent unauthorized access, which is particularly important for medical devices involving personal privacy.

The structures of these components are mostly well-known to those skilled in the art and will not be elaborated here. These components or other additional components can be reasonably configured according to different usage scenarios, which is not limited by the invention.

By integrating the aforementioned components, the human-machine interface system 400 can not only efficiently acquire and parse brain signals but also possesses flexible functional expandability and adaptability, meeting diverse clinical needs and technical application scenarios.

In a further aspect, the invention relates to the use of a conductive adhesive interface layer in implantable human-machine interfaces, wherein the conductive adhesive interface layer comprises a hydrophilic polymer and a hydrophobic polymer formed by polymerization of TPM monomers.

In yet a further aspect, the invention provides a method for treating or preventing a disease in a subject in need thereof, comprising implanting the implantable bioelectronic device according to the invention into a tissue of the subject. Preferably, the implantable bioelectronic device comprises dehydrated conductive adhesive interface layer.

The principles of the present invention are illustrated by the following examples, which are provided by way of illustration, but should not be taken as limiting the scope of the invention:

### EXAMPLES

### Methods

Animals: All experiments were approved by the Institutional Animal Care and Use Committee (IACUC) of the Chinese Institute for Brain Research (CIBR), Beijing (license number: CIBR-IACUC-049). Male Sprague Dawley rats (8-15 weeks) were obtained from Beijing Vital River Laboratory Animal Technology Co., Ltd. The animals were housed in a standard facility at the CIBR under controlled environmental conditions, including a temperature range of 21-23° C., humidity levels of 55-60%, and a 12-hour light-dark cycle. The animals were provided ad libitum access to water and food. Fresh porcine heart tissue was obtained from local butcher shops. To mitigate pain and reduce the risk of infection, meloxicam (2 mg/kg) and ceftriaxone sodium (100 mg/kg) were administered one hour before surgery and on postoperative days 0, 1, 2, 3, 5, and 7.

Preparation of PVA PTPM: The primary solution was prepared by dissolving 6 g of polyvinyl alcohol 1799 (PVA-1799, 98-99% hydrolyzed; Aladdin) in 50 mL of dimethyl sulfoxide (DMSO; Aladdin) under continuous stirring at 95° C. for 2 h. Subsequently, 0.85 mL (0.35 mL and 1.67 mL for PVA/PTPM ratios of 17:1 and 3.5:1, respectively) of 3-(trimethoxysilyl) propyl methacrylate (TPM; Alfa) and 25 mg (1.54 mg and 50 mg for PVA/PTPM ratios of 17:1 and 3.5:1, respectively) of DMPA (98%; Alfa) were added to the primary solution. The mixture was homogenized by stirring for 30 min in the dark to prevent premature photoreaction. Before assembly with μECoG, the resulting solution was defoamed by centrifugation (Cence, L500-A) at 2000 rpm for 3 min.

Fabrication of μECoG arrays: The 1024/64-channel μECoG arrays and 4-mm discs were fabricated using standard microfabrication techniques on 4-inch silicon wafers. Initially, a 3 μm layer of polyimide (P12611, HD MicroSystems) was spin-coated onto silicon wafers. The coated wafer was subjected to soft baking, followed by vacuum curing at 300° C. Photolithographic patterning was performed using an AR-N 4340 photoresist to define electrode structures. Subsequently, a 10 nm titanium layer and a 200 nm gold layer were deposited, and the excess material was removed through a lift-off process. A second 3 μm polyimide layer was spin-coated, soft-baked, and vacuum-cured. To define the electrode contours and contact pads, AZ 4620 photoresist was used as a hard mask for reactive-ion etching (RIE). After the microfabrication steps were completed, the devices were optically inspected for defects. The devices were then released from the silicon wafer in deionized water, dried using N2 blowing, and transferred to glass substrates for packaging.

For the 64-channel μECoG arrays, an Anisotropic Conductive Film (ACF) packaging method was employed. For the 1024-channel electrodes, a gold ball-bonding method was used. Both device types were integrated into custom-designed flexible flat cables (FFC). During gold ball-bonding, the electrode pad and FFC pad were precisely aligned and stacked, followed by bonding a gold ball within the electrode pad via to establish a secure connection between the electrode and FFC. For the 1024-channel electrode arrays, Molex connectors (502430-6421) were incorporated at the FFC edge, enabling reliable electrical interfacing with the corresponding Molex connectors (502426-6421) on the Intan RHD 128-channel headstage (C3316).

The electrodeposition of PEDOT:PSS was conducted in an aqueous solution containing EDOT (0.01 M) and PSS (2 wt %) using NanoZ's fixed plating time mode.

aGel-μECoG assembly and sterilization: The defoamed hydrogel solution was spread onto the μECoG arrays using a scrape-coating technique to achieve precise thickness control. The coated aGel-μECoG arrays underwent pulsed UV irradiation (365 nm wavelength, 0.8 W cm-2, 5 s On/3 s OFF) for 30 min, followed by solvent exchange in ultrapure deionized water (DI water, 18.2 MΩ·cm) for an additional 30 min. Next, aGel-μECoG was immersed in sterile PBS (1×, pH 7.4, Macklin) for three days to ensure complete elimination of residual DMSO. PBS was renewed daily. The coating thickness was measured using a Stylus Profiler (DektakXT, Bruker). Prior to implantation, aGel-μECoG arrays were dehydrated by baking at 60° C. for 15 min. The dehydrated arrays were then sealed in sterile packaging and subjected to UV sterilization for 30 min to ensure sterility.

ATR-FTIR analysis of PTPM formation: The formation of PTPM was characterized using attenuated total reflection Fourier transform infrared spectroscopy (ATR-FTIR). Spectral measurements were conducted using an FT-IR spectrometer (Bruker Equinox 55) equipped with ATR accessories. The spectra were recorded over a wavenumber range of 400-4000 cm-1.

Measurement of water contact angles: Water contact angles were measured with a contact angle meter (OCA25, Germany). Water contact angles were acquired on dry state aGel and PVA films. Water contact angles were also acquired on PI, fully swollen aGel, and PVA films to compare their hydrophilicity.

Morphological characterization via cryogenic scanning electron microscopy: The microscopic morphologies of the aGel and aGel-μECoG specimens were analyzed using cryogenic scanning electron microscopy (cryo-SEM). The specimens were transferred into a cryo-preparation chamber (LEICA EM ACE600) under vacuum conditions and subjected to sublimation at −100° C. for 20 min. Surface conductivity was achieved by platinum coating in an Ar atmosphere (15 mA, 200 s). The specimens were subsequently transferred to a microscope cryostage (S-4300, HITACHI, Ltd., Japan) maintained at −137° C. Image acquisition was performed using operating parameters of 3 kV landing energy and 10 μA current.

Tensile measurements of aGel: The mechanical properties of aGel were tested using fully swollen specimens in PBS and characterized using a universal testing machine (Instron5944 with a 1 kN load cell). Tensile tests were performed on unnotched dumbbell-type specimens (GB/T 528-2009, length: 25 mm, width: 4 mm, thickness: 1.5 mm) at a constant tensile speed of 10 mm/min. The specimens were loaded with the maximum strain until rupture. The fracture energy, which characterizes the material toughness, was determined by integrating the complete stress-strain curve and multiplying it by the initial specimen length:

$${G}_{f} = ( {\int }_{0}^{ε f} σ ( ε ) d ε ) {L}_{0}$$

Young's modulus was calculated from the linear region of the stress-strain curve within the initial 10% strain range and is given by:

$$E = \frac{σ}{ε}$$

where ε is the engineering strain, σ is the engineering stress, L₀ is the initial length, and ε_{f} is the failure strain of the specimen.

Lap shear adhesion tests for PI substrates: The adhesive performance of aGel was evaluated through lap-shear tests (ASTM F2255 standard) by examining both the PI substrate and the biological tissue interfaces. In the lap-shear adhesion test for PI, aGel was applied to one end of the PI substrate, and an area of approximately 25 mm×25 mm was covered with another PI substrate and pressed to set. The specimens were tested after soaking in PBS for three days at room temperature and characterized by a universal testing machine (Instron5944 with a 1 kN load cell). The samples were stretched at a constant tensile speed of 10 mm/min until failure.

Lap shear adhesion test for biological tissue interfaces: For the lap adhesion test at the tissue interface, the aGel was applied to one end of a stiff backing (glass) measuring approximately 25 mm×25 mm. The biological tissue (porcine heart) was firmly adhered to another piece of glass backing and kept moist by spraying with water. The aGel with backing and tissue interface was pressed together and set for 30 min before the test. Lap shear adhesion tests were performed using a mechanical testing machine (TST-01M with a 50 N load cell, PubTester). The specimens were pulled apart at a constant speed of 5 mm/min until failure. The adhesion strength of both configurations was calculated by dividing the force at failure by the overlap area.

90° peeling test for interfacial toughness: The interfacial toughness was determined using a 90° peeling test performed in accordance with ASTM D3330 standards. The test was conducted on an aGel sheet (dry state, dimensions: 30 mm in length, 15 mm in width, and 0.2 mm in thickness) adhered to a porcine heart immobilized using a glass backing. During the peeling process, the measured force reached a steady state once the peeling progressed uniformly. The interfacial toughness was calculated by dividing the steady-state peel force by the width of the sample.

Volume and mass swelling ratio measurements: The volume and mass swelling ratios were determined using dry-state aGel sheets. The initial dimensions, including length (l₁), width (l₂), height (h₀), and mass (m₀), were recorded in the dry state. The specimens were then immersed in PBS at room temperature until equilibrium swelling was reached. After equilibrium was reached, the swollen dimensions were measured: length (l₁ₛ), width (l₂ₛ), height (hₛ), and mass (mₛ).

The volume swelling ratio (Qᵥ) and mass swelling ratio (Qₘ) were calculated using the following equations:

$${Q}_{V} = \frac{{l}_{1 s} \times {l}_{2 s} \times {h}_{s} - {l}_{1} \times {l}_{2} \times {h}_{0}}{{l}_{1} \times {l}_{2} \times {h}_{0}} \times 100 \%$$

$${Q}_{M} = \frac{{m}_{s} - {m}_{0}}{{m}_{0}} \times 100 \%$$

AC conductivity measurements: AC conductivity measurements were conducted on PBS and aGel specimens equilibrated in PBS using a four-probe tester (ST2242, Suzhou Lattice Electronics) and a Gamry Reference 620 potentiostat. PBS was tested in a standardized vessel with dimensions of 4 mm (thickness), 20 mm (width), and 50 mm (length). For the aGel specimens, the individual dimensions were measured and recorded prior to testing.

The conductivity (σ) was calculated from the measured impedance (R) using the following formula:

$$σ = \frac{1}{ρ} = \frac{1}{R} \cdot \frac{L}{A}$$

where σ is the conductivity (S m⁻¹), ρ is the resistivity (Ωm), R is the impedance (Ω), L is the testing distance (probe spacing, m), and A is the cross-sectional area of the specimens (m²).

Measurement of transmittance: Light transmission across the 400-800 nm wavelength range was measured using a UV-Vis spectrophotometer (Lambda 950, PerkinElmer). Test specimens were prepared by applying aGel coatings of varying thicknesses onto glass substrates, whereas uncoated glass substrates were used as reference blanks to account for substrate effects. Note that the aGel coatings were in a dry state during the tests. For direct comparison, the average transmission over the 400-800 nm wavelength was defined and obtained from the following equation:

$$T \% = \frac{{\int }_{400}^{800} xdx}{800 - 400}$$

In vitro cytotoxicity tests: The cytotoxicity of −aGel and +aGel was evaluated following the ISO 10993-12 Standards. Films (thickness <500 m) were immersed in the culture medium at a ratio of 6 cm² mL⁻¹ and incubated at 37° C. for 24 h to prepare the extracts. NIH-3T3 cells were cultured in Dulbecco's modified Eagle's medium (DMEM) supplemented with 10% fetal bovine serum and 1% penicillin-streptomycin. Cells were subcultured upon reaching 90% confluency, seeded at 5×10³ cells per well in 96-well plates, and incubated for 24 h.

After the initial incubation, the cells were exposed to the extract medium for 24 h, while the control groups were maintained in normal medium. Cell viability was assessed using a live/dead assay with calcein-AM and propidium iodide for live and dead cells, respectively. Fluorescent images and live/dead cell ratios were obtained using an Opera Phenix™ high-throughput and high-connotation imaging analysis system. Cell viability was calculated as the ratio of the total number of live cells to the total number of cells.

Non-specific protein adsorption experiments: Non-specific protein adsorption was assessed following established protocols. PI, aGel and PVA samples were cut to standard size and equilibrated in PBS overnight. They were then incubated in a 2 mg mL-1 BSA solution at 37° C. for 90 minutes. After incubation, loosely adsorbed proteins were removed via two PBS washes, while tightly bound proteins were desorbed using ultrasonication in 1% SDS solution. The protein concentration in the desorption solution was quantified using a micro-BCA protein assay kit (Thermo Scientific, 23235).

Cell adhesion and growth experiments: The PI substrate was prepared by applying PI to the cell culture glass slides using the spin-coating method. The aGel was coated onto the PI substrate using the same spin-coating technique to produce the aGel substrate. Prior to use, the samples were sterilized using ultraviolet irradiation. After sterilization, the substrates were washed three times with PBS (3 min per wash) to remove residual contaminants. NIH3 T3 cells were seeded at a density of 3×10⁴ cells per substrate and cultured at 37° C. for 1, 3, and 5 days. Viable cell density was quantitatively assessed using calcein-AM/propidium iodide staining.

Propidium iodide staining: The electrodes were stained with propidium iodide solution (Coolaber, China, 1 μg/mL) for 7-8 min, followed by three washes with PBS solution (3 min per wash). The stained samples were imaged using a Nikon two-photon microscope operated in the single-photon mode.

COMSOL finite element model of the rat brain: To evaluate the effects of hydrogel thickness and conductivity, finite element models (FEM) of a rat brain and aGel-μECoG electrodes using COMSOL Multiphysics (Version 6.2, COMSOL Inc.) was developed. The rat brain was approximated as a multilayered spherical structure, including the gray matter, cerebrospinal fluid (CSF), dura mater, skull, and scalp. The aGel-μECoG system was modeled as a thin, flexible layer conforming to the cortical surface, comprising a 6 μm-thick polyimide (PI) substrate, PEDOT:PSS electrodes (20 μm diameter, 200 μm spacing), and an adhesive aGel layer. The thickness and electrical conductivity of each layer were assigned based on previously reported values. To optimize the computational efficiency while maintaining accuracy, a quarter-sphere model was employed, leveraging the symmetry of the rat brain. A free tetrahedral mesh was generated with a finer mesh size at the electrode-hydrogel-cortex interface to capture the fine-scale interactions. Convergence was achieved with a relative tolerance of 10⁻³. The FE models were computed using the AC/DC module. The scalp was grounded throughout the simulation.

Design optimization using computational simulations: Neuronal recording was simulated by placing radially oriented dipoles at a depth of 0.3 mm in the gray matter, with a 100 μm separation between poles and currents of +1 μA and −1 μA applied to the upper and lower poles, respectively. Using this model, the inventors systematically swept two key parameters of the aGel layer—its thickness (t) from 0 to 100 μm and conductivity (σ) from 0.01 to 10 S m⁻¹—and generated isopotential maps to analyze how these parameters influence the signal magnitude. This analysis revealed a clear trade-off between the thickness and conductivity of the aGel layer: thinner layers or lower conductivities generally preserved stronger signals, whereas thicker or more conductive layers led to greater signal attenuation. For example, an aGel coating with a thickness of t<30 μm and conductivity of σ=−2 S m⁻¹ can maintain a maximum signal amplitude exceeding 50% of the potential observed under aGel-free conditions. Spatial resolution was characterized by potential decay profiles, with the critical resolution distance (d_{c}) defined as the point where the normalized potential dropped to 0.1 of its initial value. This analysis revealed two key design principles: (1) for fixed σ, dc decreases monotonically with decreasing t, and (2) at constant t, lower a values yield smaller dc. These findings provide clear guidelines for designing aGel layers to balance the signal strength and spatial resolution in neural recording applications.

For electrical stimulation, the inventors investigated the effects of aGel parameters on the voltage distribution within the brain by applying a constant 1 V voltage between adjacent electrodes. The voltage distribution was quantified to determine how effectively the applied energy was transferred through the aGel layer into the surrounding neural tissue. To optimize the aGel design, the inventors introduced an energy transfer ratio (ΔV1/ΔV2), defined as the ratio of the potential obtained in the brain tissue below the electrodes to the potential applied to the electrodes. An energy transfer ratio of 0.5 was chosen in our analysis as the threshold for efficient stimulation, balancing the energy delivery to the brain while minimizing the losses within the aGel. Through this analysis, the inventors determined that the optimal aGel parameters for efficient stimulation were a thickness of 1-10 μm and a conductivity range of 0.1-2 S m⁻¹.

Integrating these findings, the inventors identified an ultrathin aGel layer (~10 μm thickness, ~2 S m⁻¹ conductivity) as the optimal design for balancing high-fidelity recording and stimulation efficiency.

In vitro electrical characterizations: The single-frequency impedance at 1 kHz was measured using an Intan RHD 1024-channel recording controller and RHD 128-channel headstage, with a platinum electrode as the reference. CV and EIS were performed using a Gamry Reference 620 potentiostat with a platinum counter electrode and an Ag/AgCl reference electrode. CV measurements were conducted at a scan rate of 100 mV/s within a voltage range of −0.5 V to +0.5 V. The EIS data were acquired over a frequency range of 1 Hz to 100 kHz using an AC voltage amplitude of 25 mV.

Aging experiments: The devices were aged by immersion in PBS at 60° C. CV, EIS, and impedance measurements were performed at specific time points (0, 2, 4, 6, 8, 10, 12, 14, 21, and 28 days) and subsequently every two weeks or one month.

Animal surgery for functional cortical mapping: Sprague Dawley rats (350-500 g, 10-15 weeks) were used for acute functional cortical recordings. Anesthesia was induced using 3-5% isoflurane and maintained at 1.5-3%. The scalp and right thigh were shaved, and the skin was disinfected with iodine and alcohol. To minimize pain and bleeding, 1% lidocaine (15 mg/kg, SC) and etamsylate (125 mg/kg, IP) were administered. Brain swelling was prevented by intravenous injection of 20% mannitol (2 g/kg, IV). Prior to signal recording, the rats were transitioned from isoflurane anesthesia to an intraperitoneal injection of 5% ketamine (100 mg/kg) and 10% xylazine (10 mg/kg). Supplemental anesthetic doses were administered every 30 min.

A midline scalp incision was made to expose the skull, and the left temporalis muscle was excised. A square craniotomy (AP: 2.5 mm to −6 mm; ML: +1 mm to +7 mm) was performed using a stereotaxic apparatus (RWD Life Science, China). The bone flap was carefully removed, and the dura mater was excised. The exposed brain surface was kept moist with a saline-soaked gelatin sponge, and bleeding was controlled prior to electrode placement.

For sciatic nerve preparation, a small incision was made on the contralateral thigh, and the connective tissues around the nerve were dissected. An insulated plastic sheet was placed under the nerve to prevent direct muscle stimulation, and a bipolar electrode was attached.

Bipolar electrical stimulation and neural recordings for functional cortical mapping: Bipolar electrical stimulation of the sciatic nerve was performed using two disposable needle electrodes (Haizhen, EN-ST2000; length: 13 mm, diameter: 0.40 mm). The cathode was then placed close to the spinal cord. A signal generator (DG4202, RIGOL) delivered 1 Hz square-wave triggers to the Intan RHS 128-channel stimulation/recording controller (M4200, Intan Technologies) and RHS 16-channel stim/record headstage (M4016) to generate biphasic current pulses. The stimulation parameters were biphasic, cathodic-first, 1 ms phase duration, and 1 Hz frequency. The stimulation intensity were 50, 100, 200, 500, or 1000 μA.

Neural signals were recorded using an INTAN 1024-channel recording controller and eight RHD 128-channel headstages, at a sampling rate of 10 kHz. Three device configurations (μECoG, 10-μm aGel μECoG, and 100-μm aGel μECoG) were tested for each rat. For each configuration and stimulation intensity, 1-min recordings were obtained. In addition, 5-min resting-state signals were recorded for each electrode configuration.

Data recording and processing for functional cortical mapping: Neural signals were preprocessed using a 4th-order bidirectional Butterworth bandpass filter (4-1000 Hz) and downsampled to 2000 Hz. Signals were segmented into individual trials, each spanning from a 0.5 s pre-stimulus to a 0.5 s post-stimulus. A trial was classified as “good” if the maximum amplitude within the post-trigger window (0-0.5 s) exceeded 1.5 times the mean amplitude of the pre-trigger baseline window (−0.5-0 s) (0.5 s duration, threshold multiplier=1.5). Channels with impedance >10 MΩ or a good trial rate (ratio of good trials to total trials) of <10% were excluded from the study.

Baseline correction was applied by subtracting the pre-stimulus means, and trial-averaged responses were visualized for SSEP characterization. The inventors used good-trial-averaged and Hilbert-transformed signals to compute the SNR, which was quantified as the root mean square (RMS) of the signals within the post-stimulus window (0-100 ms) and normalized to the RMS of the pre-stimulus baseline window (−100-0 ms):

$$SNR = {RMS}_{{t}_{0}}^{{t}_{100}} / {RMS}_{{t}_{- 100}}^{{t}_{0}}$$

An adaptive re-referencing pipeline was implemented to suppress common noise. Initial SNR maps were generated from good trials using a sliding thresholding window (0.2 s duration, threshold multiplier=2), which guided the subsequent two-dimensional Gaussian fitting. These maps were then fitted using a two-dimensional Gaussian function as follows:

$$G ( x , y ) = A \cdot {e}^{- ( \frac{{( x - {x}_{0} )}^{2}}{2 {σ}_{x}^{2}} + \frac{{( y - {y}_{0} )}^{2}}{2 {σ}_{y}^{2}} )}$$

where A is the amplitude, (x₀,y₀) represents the centroid of the SNR maxima, and σₓ,σ_{y} denote the spatial spread along the x- and y-axes, respectively. The farthest 100 channels from the Gaussian centroid were selected for re-referencing. The SNR maps were subsequently recalibrated using the re-referenced signals.

Frequency-resolved SNR mapping (FIG. 15) employed 4th-order Butterworth bandpass filtering across canonical frequency bands (θ:4-8 Hz, α:8-12 Hz, β:12-30 Hz, low γ:30-70 Hz, and high γ:70-190 Hz).

For the stimulation intensity analysis (FIG. 14), spatially localized channels were selected from the high γ-band (70-190 Hz) SNR map by retaining those with Gaussian-fitted values ≥90% of the maximum. The mean amplitude of the raw signals (prior to the Hilbert transformation) within these channels was calculated to quantify the stimulation-induced response. Post-stimulus Hilbert-transformed root mean square (RMS) amplitudes (10-200 ms) were normalized to those of aGel-μECoG under 1 mA stimulation.

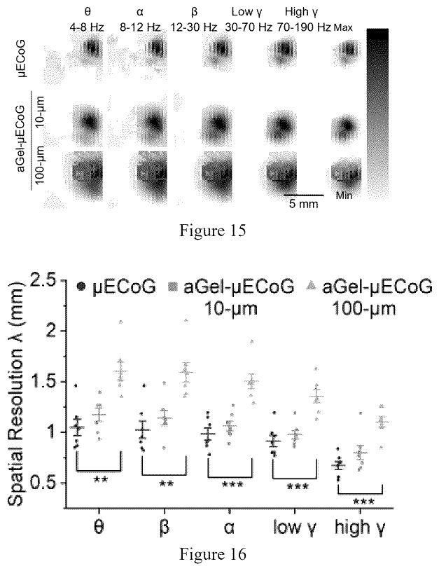

Resting-state magnitude squared coherence analysis (FIG. 16) used signal bandpass filtered between 1-1000 Hz, 50 Hz notch filtering, and adaptive common average re-referencing. Continuous 5-second epochs were segmented and analyzed for frequency-specific coherence across various frequency bands using the multitaper method to estimate magnitude-squared coherence. Coherence-distance relationships were modeled with an exponential decay function: y=a·exp(−x/λ)+c to derive an approximation of the spatial resolution λ, where the spatial resolution parameter λ represents the characteristic decay length of coherence (smaller λ values indicate stronger spatial localization). The model parameters (a, λ, and c) were optimized via nonlinear least-squares fitting.

Immunohistochemical analysis: To evaluate the biocompatibility of the +aGel and −aGel films, 4-mm diameter discs for brain implantation were prepared according to the ISO 10993-6 standards. To distinguish between the anterior and posterior disc surfaces, an “F” mark was added owing to the transparency of the materials.

Surgical procedure for histological analysis: Sprague Dawley rats (250-350 g, 8-10 weeks) were anesthetized with isoflurane and positioned in the stereotaxic apparatus (RWD Life Science, China). Anesthesia and pain management were performed according to the previously described protocol. A circular craniotomy window with a diameter of 4.4 mm was created, centered at AP −3.2 mm and ML+2.9 mm, with reference to the bregma. The films were then purified, sterilized, and implanted subdurally. The removed bone flap was returned to its original position, and Kwik-Sil (World Precision Instruments Inc., USA) was used to fill the gaps between the cranial bone. After curing the Kwik-Sil, dental cement was applied to provide additional reinforcement. The skin was then sutured.

Tissue collection and processing for histological analysis: At specified time points, the rats were anesthetized with urethane (1.5 g/kg) and perfused transcardially with PBS until the liver became bloodless. Subsequently, 10% formalin solution was perfused until the body stopped trembling. The brains were removed, immersed in 10% formalin solution overnight, and dehydrated in 30% (w/v) sucrose solution the following day. Brains were coronally sectioned and mounted on a sample holder using an optimal cutting temperature (OCT) compound (Tissue-Tek, USA). The sections were cut into 25 μm slices using a cryostat (Leica CM3050 S, Germany), mounted on glass slides, and stored at −80° C.

Immunostaining protocol: Brain slices were washed three times with PBS (10 min each) and blocked for 1.5 h in 5% (w/v) bovine serum albumin (BSA) prepared in 1×PBST (0.5% Triton in 1×PBS). After blocking, the BSA solution was removed, and the slices were incubated overnight at 4° C. with the following primary antibodies: rabbit anti-GFAP (1:500, Bioss #bs-0199R-1, USA), goat anti-Iba1 (1:500, Abcam #ab289874, USA), and rat anti-NeuN (1:500, Abcam #ab279297, USA). After primary antibody incubation, the slices were washed three times with PBS (10 min each) and incubated at room temperature for 1.5 h with the following secondary antibodies: Alexa Fluor 546 donkey anti-rabbit (1:1000, Invitrogen #2411581, USA), Alexa Fluor 488 donkey anti-goat (1:1000, AffiniPure #705545147, USA), and Alexa Fluor 647 donkey anti-rat (1:1000, Jackson ImmunoResearch #712-605-153, USA), while avoiding exposure to light. The slices were then washed five times with PBS (10 min/wash). Finally, they were mounted using an anti-fading mounting medium containing 4′,6-diamidino-2-phenylindole (DAPI; Beyotime Biotechnology, China), stored at −20° C., and protected from light exposure.

Fluorescence imaging and analysis: Fluorescence imaging was performed using a VS200 Virtual Slide Microscope (Olympus, Japan) and analyzed using QuPath software (Queen's University Belfast, UK). The threshold and smoothing values were set in the “Thresholder” tool to identify the brain slice boundaries. Subsequently, the cortical regions were delineated using a custom-written script, allowing manual adjustments of the curve range to enhance recognition accuracy. Owing to the high density of GFAP-labeled astrocytic processes and the difficulty in identifying cell bodies, the inventors assessed the proportion of GFAP-positive areas in various regions. By contrast, NeuN-labeled neurons had clear boundaries, allowing the quantification of the proportion of NeuN-positive cells. For Iba1-labeled microglia, the inventors distinctly identified cell bodies and defined co-localization with DAPI as positive Iba1 expression, calculating the proportion of Iba1-positive cells in various regions.

For GFAP analysis, regions of GFAP expression were classified as positive, whereas all other areas were deemed negative, and the highlighted edges of the brain slices were excluded from the area calculations. For NeuN and DAPI, “Cell Detection” was used to identify individual cells. Data from the implanted side were normalized to the control side to account for individual variability.

SHG imaging: SHG images were obtained using a two-photon upright microscope (Nikon). The excitation wavelength for the SHG signal was 920 nm, and the detected light wavelength ranged from 457 to 463 nm. Additionally, a non-specific fluorescence detection band was established to exclude interference from autofluorescence, with an excitation wavelength of 1040 nm and a received light wavelength of 564-629 nm.

H&E analysis: Brain tissue specimens were fixed in 10% neutral-buffered formalin, followed by sequential gradient ethanol dehydration, xylene clearing, and paraffin embedding. Tissue sections (3-5 μm thick) were prepared using a rotary microtome. Prior to staining, the sections were dewaxed in xylene and rehydrated with a descending ethanol gradient. H&E staining was performed using standardized protocols, after which the sections were dehydrated using an ascending ethanol series and cleared in xylene. The stained sections were permanently mounted with a neutral resin mounting medium (Solarbio, China) and imaged under bright-field illumination using a Leica DM3000 microscope (Leica Microsystems, Germany).

Animal surgeriesfor chronic study: Sprague Dawley rats (250-300 g, 8-9 weeks) were used for chronic recordings. Ten animals were divided into two groups: the control group (n=5) was implanted with μECoG arrays, and the experimental group (n=5) was implanted with aGel-ECoG arrays. Animals were handled for two days prior to surgery for acclimatization. Anesthesia and pain management were performed according to the protocols described previously. After lidocaine application and tissue clearance, four self-tapping screws were inserted into the skull to anchor dental cement (Sun Medical, Japan). A cranial window was drilled using a 0.5 mm drill bit (RWD Life Science, China) at coordinates relative to bregma: AP: −3.5 mm to −8 mm; ML: +0.5 mm to +5.5 mm. A screw electrode positioned at AP: −10 mm, ML: 0 mm was used as the ground and reference for electrical recording. The 64-channel aGel-μECoG and μECoG were placed on the visual cortex based on the rat brain atlas, and the bone flap was replaced over the cranial window. The skull was sealed using Kwik-Sil (World Precision Instruments Inc., USA) and dental cement. A stud was fixed to the skull for head stabilization during awake recordings. A custom 3D-printed cap was used to protect the connectors.

Chronic in vivo EIS experiments: EIS was performed on 64-channel aGel-μECoG and μECoG electrodes implanted using the same surgical procedure as described earlier. Measurements were taken at 1, 3, 6, 9, 15, 22, 30, 37, and 60 days post-implantation. Testing was conducted using a Gamry potentiostat with a cranial screw serving as both the reference and counter electrodes. The EIS measurement parameters were the same as those used for the in vitro EIS measurements.

Equivalent circuit model: The in vitro and in vivo EIS analyses used different equivalent circuit models, as shown in FIG. 21. The in vitro EIS model comprised the solution resistance (R_{S}), electrode-tissue interface resistance (R_{E}), and a Constant Phase Element (Z_{CPE}). Here, R_{S} represents the access impedance of the circuit, R_{E}, and the Z_{CPE} collectively modeled at the electrode-tissue interface. In contrast, the in vivo EIS equivalent circuit incorporated two additional elements, C_{T} and R_{T}, which represent the capacitive and resistive components of the tissue, respectively. Note that the equivalent circuit components of the aGel layer contributed minimally, and were not considered for simplicity. EIS was fitted using Gamry Echem Analyst with the Simplex Method algorithm, setting the Maximum Iterations to 300. The initial value ranges for R_{S} and R_{E} were determined based on the in vitro EIS data and the corresponding equivalent circuit model. These initial values were subsequently applied to fit the in vivo EIS data using the in vivo equivalent circuit model, enabling the extraction of the values for the remaining circuit elements. The impedance of the Z_{CPE} is defined as:

$${Z}_{CPE} = \frac{1}{{{Y}_{0} ( j ω )}^{α}}$$

where Y₀ is the Z_{CPE} constant, j is the imaginary unit, ω is the angular frequency, and Z_{CPE} is the Z_{CPE} exponent, which ranges from 0 to 1. The Z_{CPE} functions as an ideal capacitor when α=1, and as an ideal resistor when α=0. The capacitance was calculated using the equation provided above.

1 kHz impedance analysis: To characterize the change of electrode impedance over the days of implantation, the inventors employed a generalized additive model (GAM) to identify breakpoints and subsequently modeled each phase using linear regression. Only the electrodes with impedance values between 1 kΩ and 1 MΩ were included in the analysis. The impedance changes over time were divided into three distinct phases, separated by two breakpoints. The first breakpoint was defined as the maximum impedance on the fitted curve, and the second breakpoint was defined as the intersection of the curve with the mean plus the interquartile range (IQR) of the impedance values measured after 60 days, when stabilization occurred. Each phase was modeled using linear regression to quantify the relationship between the implantation duration and impedance. All analyses were performed using the statsmodels library in Python.

SSVEP data recording: The day of surgery was designated as day 0. The SSVEP consisted of a signal generator (DG4202, RIGOL) that controlled the white-light LED. The signal generator was configured to produce an 8 Hz square wave (3 V high, 0 V low, 50% duty cycle). The LED emitted a luminous intensity of 32,000 cd/m² and was positioned 10 cm away from the rat eyes. The output from the generated signal was connected to a BNC T-adapter to synchronize the signal between the LED and INTAN recording system.

To minimize movement artifacts, the rats were restrained using a custom-designed jacket that stabilized their bodies and lifted them slightly. A stud fixed to the skull during surgery was used for head fixation, eliminating the interference caused by head movements. Two days before the recording, the animals were habituated to the setup by placing them in a jacket. Each recording session consisted of a 10-second rest-state recording followed by a 3-min SSVEP recording. To confirm that the recorded signals originated from brain activity, an eye-covered test was conducted. During this test, the rats' eyes were covered with an eye mask, and the recording procedure was repeated as described above.

SNR analysis for SSVEP: Fast Fourier Transform processing was performed on the recorded SSVEP signals from 10 s to 180 s. The SNR was computed using the following formula:

$$SNR = \frac{( n - 1 ) \cdot P ( {f}_{t} )}{( {\sum }_{f = {f}_{1}}^{{f}_{2}} P ( f ) ) - P ( {f}_{t} )}$$

where P(f) denotes the power calculated by FFT across frequencies f₁=7 Hz to f₂=9 Hz, fₜ is the target stimulation frequency equal to 8 Hz, and n represents the number of frequency bins between f₁ and f₂. To mitigate transient artifacts, epochs containing signal amplitudes exceeding 2 mV were considered as artifacts and were automatically removed.

Mixed-effects regression modeling of SSVEP: To analyze the signal stability during long-term implantation, the inventors employed LMM to account for the complexity of in vivo data recording. Random effects were included to capture the variability across individual rats, electrode channels, and other unintended sources of variation. The LMM was designed to estimate the fixed effects of implantation time (Time in week), electrode type (ElectrodeType), and their interaction (Time×ElectrodeType) on SNR, while minimizing the influence of random effects. The model is formulated as follows:

$$Y = {β}_{0} + {β}_{1} \cdot ElectrodeType + {β}_{2} \cdot Time + {β}_{3} \cdot ( ElectrodeType \times Time ) + μ + v + ε$$

Here, Y represents the SNR (on a linear scale); β₀ represents the intercept, corresponding to the baseline SNR (i.e. Time=0) for the aGel-μECoG electrodes (i.e., the reference group); β₁ represents the difference in baseline SNR between the μECoG and aGel-μECoG electrodes; β₂ represents the temporal trend in SNR over time for the aGel-μECoG electrode; and β₃ represents the interaction effect, capturing the differences in time-dependent trends between the μECoG and aGel-μECoG electrodes. The random term μ represents the random effect for each rat, accounting for subject variability, and v represents the random effect for each electrode, capturing electrode variability. The residual term F represents the unexplained error or noise that follows a normal distribution. Finally, the SNR retention rate was determined by calculating the ratio of the predicted values at each time point to those from the first week.

Statistical analysis: All statistical analyses were performed using Origin 2021. The Kolmogorov-Smirnov test was used to assess the normality of data distribution. For cytotoxicity and cell adhesion experiments with a normal distribution, one-way ANOVA was conducted; otherwise, the Kruskal-Wallis test was used for significance analysis. For electrophysiological data, two-way ANOVA with Tukey's multiple comparison test and two-tailed unpaired t-test were used. For in vivo histological analysis and EIS experiments that were normally distributed, a two-tailed unpaired t-test was conducted; if the data were not normally distributed, the Mann-Whitney test was used. The significance thresholds were set at ns p>0.05, * p<0.05, ** p<0.01, and *** p<0.001. Data are presented as mean±SEM, unless otherwise specified.

### Example 1. aGel Preparation and Characterizations

The aGel was engineered by combining PVA and PTPM to form a unique hydrophilic/hydrophobic heteronetwork structure (FIG. 5). As shown in FIG. 5, the initial solution system comprised DMSO as a solvent, with PVA and TPM thoroughly dissolved and homogeneously mixed (i). Then, the TPM monomers underwent complete polymerization under UV irradiation in the presence of photoinitiator (ii) before a solvent exchange step (iii), in which DMSO was displaced by DI water.

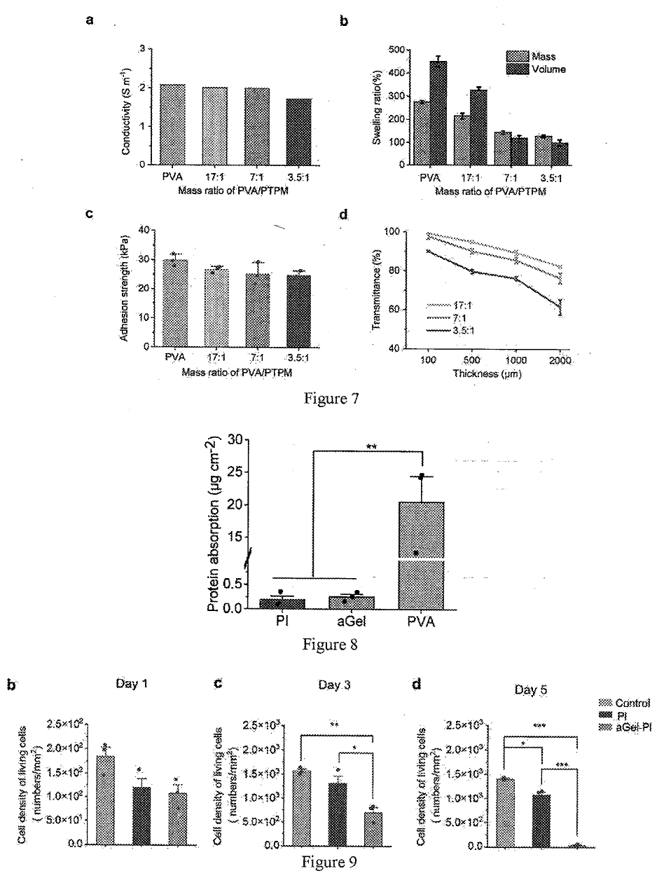

PVA was chosen as the primary hydrogel matrix because of its biocompatibility and bioadhesiveness. The incorporation of hydrophobic PTPM restricted excessive swelling of the PVA network. This design ensured structural stability while maintaining favorable adhesion properties. The successful synthesis of PVA/PTPM was confirmed by FTIR spectroscopy (FIG. 6). The ratio of PVA to PTPM significantly influences the hydrogel's functional properties. To identify the optimal composition, three ratios (17:1, 7:1, and 3.5:1) were evaluated based on key performance metrics, including ionic conductivity, tissue adhesion, swelling resistance, and transparency (FIG. 7). Among these, the 7:1 ratio was chosen for aGel in this disclosure, which exhibited superior performance across multiple functionalities.

Due to the incorporation of hydrophobic PTPM, the hydrated aGel achieved intermediate surface hydrophilicity between that of PI and hydrated PVA. Tensile tests of the aGel film demonstrated exceptional mechanical properties, including high stretchability (tensile strain: 555.1±25.8%) and toughness (fracture energy: 31.4±3.3 kJ/m2). The aGel exhibited an ultrasoft Young's modulus of 109.4±19.5 kPa, effectively bridging the mechanical mismatch between the soft brain tissue and the rigid polyimide substrate.

A significant challenge for hydrogel interfaces during long-term implantation is buckling or wrinkling caused by excessive swelling, which can lead to delamination from biological tissues or bioelectronics. Swelling tests revealed stable mass and volume swelling ratios (143.8±6.3% and 118.1±11.9%, respectively) after 180 min in phosphate-buffered saline (PBS, 1X, pH 7.4), indicating the stability of the aGel film in physiological environment. To further demonstrate the long-term stability of aGel, the film was immersed in PBS for one month and observed no significant changes in mass or volume (data not shown). The swelling resistance of the aGel layer prevents surface delamination and ensures structural integrity during long-term implantation.

Next, the in vitro biocompatibility of aGel films were assessed. Since non-specific protein adsorption is a key factor in fibrosis initiation, PVA, aGel and PI were disposed in a bovine serum albumin (BSA) solution. It was found that both aGel and PI demonstrated excellent resistance to protein fouling compared to PVA (p<0.01, FIG. 8). Next, NIH3T3 cells (a mouse embryonic fibroblast cell line) were used to conduct cytotoxicity assay and neither aGel nor PI was found to exert significant toxicity compared to the negative control medium. Further, the ability of aGel and PI to support the adhesion and growth of NIH3T3 cells was assessed. The experimental results indicated that during the initial 24-hour culture period, cell attachment density was low on both PI and aGel surfaces (FIG. 9). On day three, there was a significant decline in cell density on aGel surface (p<0.05). The results indicate that aGel has a low protein adsorption and weak capacity for cell adhesion and growth, highlighting its exceptional anti-fouling performance.

In addition, the aGel having a thickness of ≤500 μm (within the practical application range) maintained a high visible light transmittance exceeding 85% across wavelengths of 400-800 nm, enabling potential optogenetic applications. Its ionic conductivity (1.99 S m⁻¹) after soaking in PBS (1×) closely matched that of CSF, supporting its compatibility with electrophysiological applications.

### Example 2. aGel-μECoG Preparation and Characterizations

To achieve high flexibility and biocompatibility, 6 μm-thick μECoG arrays were fabricated using biocompatible PI as the substrate, with a 10 nm/200 nm Ti/Au metal layer for electrical conductivity (FIG. 3). The electrochemical performance of the 64/1024-channel electrodes was further optimized by electroplating with PEDOT:PSS.

For seamless device integration, an aGel precursor was applied to the μECoG array using a scrape-coating technique to ensure a uniform and precise layer thickness, followed by a two-step in situ gelation process involving photopolymerization and solvent exchange. The gelation kinetics were well controlled to prevent strain-induced deformation or delamination between the aGel and μECoG devices. Impedance and cyclic voltammetry (CV) measurements showed negligible changes after coating, with no observable wrinkles on the PEDOT:PSS layer. The heteronetwork structure of the aGel physically interlocked with the μECoG surface, providing robust adhesion, as confirmed by lap-shear tests, which indicated an adhesion strength of 123.5±11.2 kPa. Cross-sectional cryo-SEM revealed seamless integration at the aGel-PI interface, eliminating delamination or air gaps during chronic implantation (FIG. 10). Cross-sectional scanning electron microscopy image of the aGel-coated PI interface revealed intimate interfacial contact between the aGel coating and the PI substrate (FIG. 10, (a)). Internal morphology and structure of aGel shew a uniform, loose, and porous microstructure and network organization (FIG. 10, (b)).

To address the adhesion challenges on slippery, wet biological tissues, the inventors employed a dry cross-linking mechanism. Upon contact with wet tissues, aGel-μECoG in the dry state rapidly absorbed interfacial water through its hydrophilic PVA network. This process immediately forms strong hydrogen bonds with the tissue surface. The PTPM network further enhanced the structural stability and biocompatibility, achieving an adhesion strength of 25.2±3.8 kPa to biological tissues. A 90-degree peeling test revealed a low peeling energy (27.79±2.24 Pa) for aGel-μECoG. Ex vivo tests performed on the rodent brain and porcine heart tissue further confirmed that aGel-μECoG achieved strong adhesion and conformal contact and ensured safe removal without causing damage to diverse tissues.

Although high-density μECoG arrays are known for their superior signal quality and spatiotemporal resolution, integrating an aGel layer introduces design challenges owing to the increased source-electrode distance, potentially compromising the fidelity of weak neural signals. To address this challenge, the design parameters of the aGel layer was systematically optimized using computational simulations. The inventors constructed a five-layer rodent head model based on previous studies, including gray matter, CSF, dura mater, skull, and scalp, for finite element (FE) simulations (COMSOL Multiphysics V6.2). A 16×16 aGel-μECoG array, with 20 μm electrode diameters and 200 μm pitches, was placed subdurally in the rodent brain model in the simulations. The results revealed trends consistent with theoretical volume conduction models. Specifically, both the thickness and conductivity of the aGel layer significantly influenced the signal propagation, with thicker or more conductive layers leading to reduced signal communication efficiency (FIG. 11). Integrating the findings, the inventors identified an ultrathin aGel layer (~10 μm thickness, ~2 S m⁻¹ conductivity) as design parameters for high electrical fidelity. The fabrication processes further confirmed the feasibility of achieving ultrathin aGel layers as thin as 10 μm, validating their suitability as operational windows for aGel-μECoG arrays.

### Example 3. aGel-μECoG Preserves Signal Fidelity in High-Resolution Functional Cortical Mapping

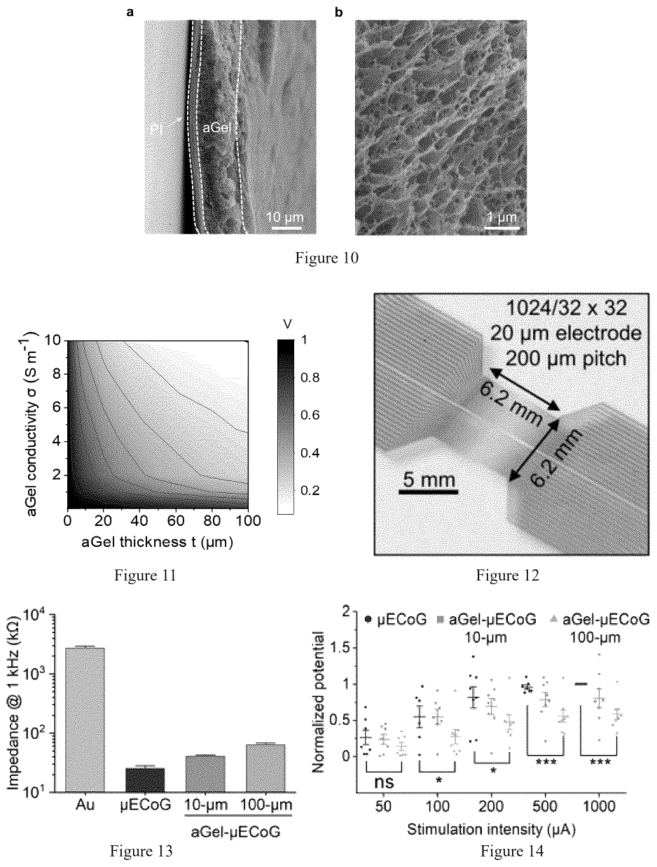

To experimentally evaluate the signal fidelity of the ultrathin aGel-μECoG arrays, the inventors performed functional cortical mapping in anesthetized rats using high-density 1024-channel devices. The arrays were fabricated on a 6-μm-thick PI substrate with 20 am diameter electrodes spaced at a 200 μm pitch and electroplated with PEDOT:PSS to reduce the electrochemical impedance (FIG. 12). To test this hypothesis and validate the FE simulation results regarding the electrical fidelity properties of an ultrathin aGel interfacial layer, three device configurations were prepared: one without aGel (ECoG), one with 10 μm aGel (10-μm aGel-ECoG), and one with 100 μm aGel (100-μm aGel-μECoG). Electrical characterization showed that the PEDOT:PSS coating effectively reduced the impedance and enhanced the charge storage capabilities across all configurations (FIG. 13). Although the addition of 10-μm and 100-μm aGel layers slightly increased the impedance due to the lower conductivity of aGel compared to that of PBS, the values remained below 100 kΩ, ensuring negligible distortion when coupled with high input impedance amplifiers.

For a fair comparison, perfusion holes were incorporated into the μECoG devices to displace the CSF and minimize the source-electrode distance. The devices were sequentially placed on the cortical surface of anesthetized rats to record signals from the left primary somatosensory cortex. Somatosensory evoked potentials (SSEPs) were elicited by stimulating the right sciatic nerve with 100 ms biphasic pulses using a pair of 26-gauge bipolar needles. The recordings revealed highly localized, large-amplitude SSEPs with peak responses at 20-30 ms post-stimulation, which is consistent with previous studies. Trial-averaged SSEPs from a representative measurement showed that the amplitude increased proportionally to the stimulation intensity for all the device configurations (FIG. 14). An unpaired t-test applied to the normalized SSEP potential for each stimulation intensity revealed a significant difference for the 100-μm aGel-μECoG, starting at a stimulation current of 100 μA, indicating the threshold for a discernible response (FIG. 14). Further analysis across all stimulation conditions showed that aGel thickness significantly influenced signal power (two-way ANOVA: F(2,15)=14.98, p=2.6×10⁻⁶). Post hoc Tukey's tests indicated no significant difference between μECoG and 10-μm aGel-μECoG (p=0.42). By contrast, the 100-μm aGel-μECoG exhibited a significantly reduced signal power (p<0.001) (FIG. 14). This observation is consistent with computational modeling predictions that thick aGel layers can attenuate signal propagation.

To assess the impact of aGel thickness on spatial resolution, the bandpass-filtered spatial maps of the SNR were analyzed (FIG. 15). These maps revealed distinct activity patterns, particularly in the gamma (γ, 30-70 Hz) and high-gamma bands (high γ, 70-190 Hz), demonstrating the ability of high-density aGel-μECoG to record high-frequency oscillations. Notably, high-gamma band activation is strongly associated with spiking activity and exhibits high spatial specificity. FIG. 15 showed that both μECoG and 10-μm aGel-μECoG maintained sub-millimeter spatial resolution at the gamma and high gamma bands, whereas 100-μm aGel-μECoG showed a broader spatial spread. The inventors further quantified the spatial resolution using magnitude-squared coherence across increasing inter-electrode distances at different frequency bands using data recorded in the resting state (FIG. 15). The data were fitted to an exponential decay model, y=a·exp(−x/λ)+c, where λ represents an approximation of the spatial resolution. An unpaired t-test applied to the spatial resolution for each frequency band revealed a significant difference for the 100-μm aGel-μECoG, indicating signal dispersion across all examined frequency bands (FIG. 16). A two-way ANOVA confirmed that the aGel thickness significantly affected the spatial resolution (F(2,15)=42.77, p<0.001). While the 10-μm aGel-μECoG maintained a resolution comparable to that of μECoG (p=0.41), the 100-μm aGel-μECoG showed a significantly degraded spatial resolution (p<0.001).

These experimental results align well with the previously proposed design principles, providing robust validation of the simulation methodology. They also demonstrated that 10 μm aGel-μECoG arrays could achieve high-resolution functional mapping comparable to high-density ECoG, validating their potential for high-fidelity neural recordings.

### Example 4. Histological Analysis Reveals Superior In Vivo Biocompatibility of aGel Coating

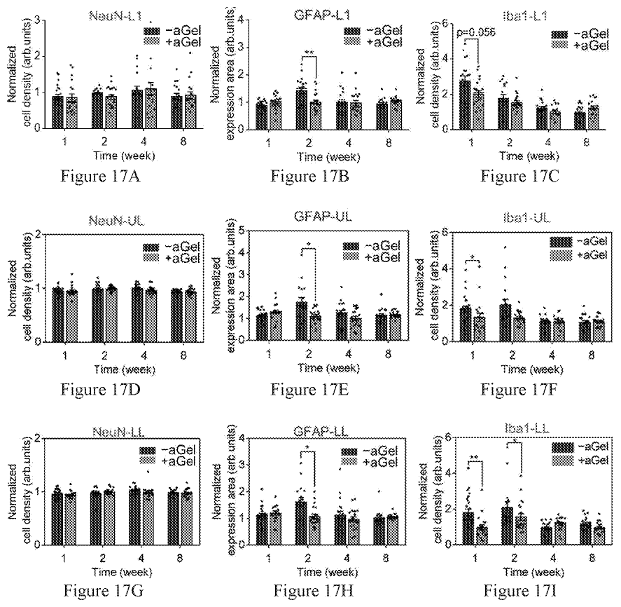

The in vivo biocompatibility of the aGel-coated devices in rat models was evaluated using chronic subdural implantation. To minimize adverse tissue reactions associated with connector depression and implant shape, the inventors fabricated 4-mm disc-shaped devices in accordance with the ISO 10993-6 standards for the biological evaluation of medical devices (FIG. 3). Two device configurations were compared, one without aGel coating and one with a 10-μm-thick aGel coating (referred to as −aGel and +aGel, respectively) to distinguish them from the 64-channel long-term implanted devices. Brain samples were collected at 1, 2, 4, and 8 weeks post-implantation. Bright-field imaging revealed no visible cortical damage in either group. To assess neuroinflammatory responses in the brain tissue underneath the implants, the inventors used immunofluorescence markers for neurons (NeuN), astrocytes (GFAP), and microglia (Iba1). The expression levels were normalized to those of the contralateral controls to minimize individual variability. To account for regional differences in cellular responses, the cortex was divided into three regions: layer I (L1), upper layers (UL, layers II-IV), and lower layers (LL, layers V-VI), based on neuronal density and morphology. NeuN staining revealed no significant difference in neuronal density between the −aGel and +aGel groups at any stage of the study (FIGS. 17A, 17D and 17G). Quantitative analysis revealed that GFAP expression, which is indicative of astrocytic activation, was significantly higher in all cortical regions of the −aGel group at two weeks post-implantation (FIGS. 17B, 17E and 17H). Similarly, Iba1 expression, a marker of microglial activation, was significantly elevated in the −aGel group compared to the +aGel group at one week in all cortical regions and at two weeks in the LL layer (FIGS. 17C, 17F, and 17I). After four weeks, no significant differences in marker expression were observed between the two groups. These results demonstrate the excellent biocompatibility of aGel-μECoG devices, characterized by weaker in vivo immune responses than those of bare μECoG devices.

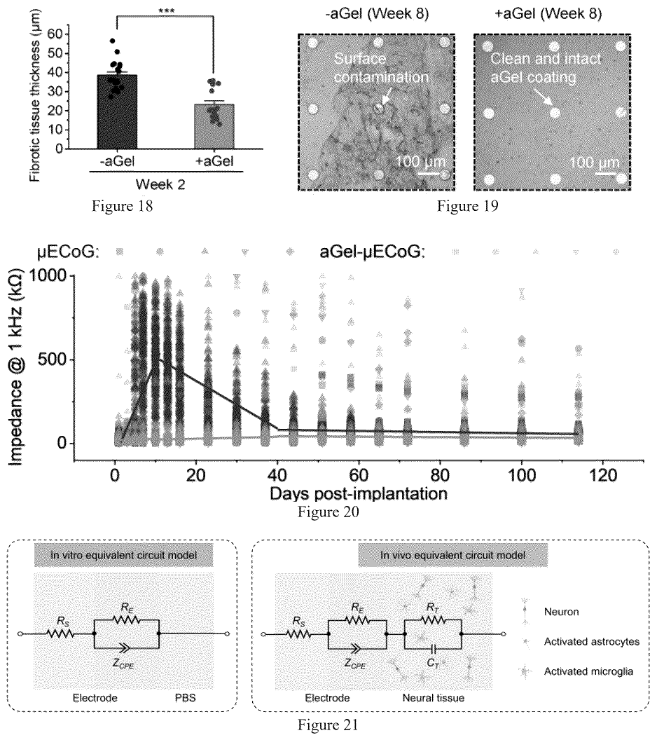

To evaluate the degree of fibrotic tissue growth, the collected tissue samples were examined using hematoxylin and eosin (H&E) staining. The −aGel implants showed substantial fibrous capsule formation by week 2 (FIG. 18). Notably, loose connective tissues formed at the brain surface in the −aGel group as early as two weeks, consistent with a previous study. The fibrous tissue encapsulation of the explanted devices was also examined using second-harmonic generation (SHG) imaging. A strong SHG signal and foreign material visible under an optical microscope were observed on −aGel discs, indicating significant collagen accumulation and protein adsorption. By contrast, no such signals were detected on the +aGel discs, indicating superior resistance to fibrotic capsule formation. Microscopic observations further confirmed that the aGel coating on explanted+aGel devices maintained its structural integrity across all time points. A representative image from week 8 is shown in FIG. 19 (right panel). Additionally, a significant amount of adsorptive contamination was observed in the −aGel devices, whereas the +aGel devices remained clean (FIG. 19). These results collectively demonstrate the in vivo capabilities of aGel-ECoG in mitigating neuroinflammation and reducing fibrous tissue formation in the brain.

### Example 5. Long-Term Impedance Stability of aGel-μECoG for Subdural Implantation

Electrochemical impedance spectroscopy (EIS) is a valuable tool for monitoring electrode-tissue interface dynamics, with established correlations between in vivo impedance spectra and histological changes. Previous studies on long-term surface electrode implantation have reported an initial rapid impedance increase followed by gradual stabilization over months to years, reflecting the progression of tissue responses from acute to chronic phases. To evaluate the long-term stability of the aGel-μECoG, the inventors employed 64-channel μECoG arrays and chronically implanted them in the visual cortex of rats. Two configurations were compared: ECoG without aGel and aGel-μECoG with a 10-μm interfacial aGel layer. Electrical characterizations confirmed that the aGel layer minimally affected the electrical properties of the PEDOT:PSS-electroplated μECoG. Accelerated aging tests in 60° C. PBS (1 ×, pH 7.4) for 100 days predicted an equivalent functional lifetime exceeding one year for both configurations, highlighting the durability of the PEDOT:PSS layer and aGel coating under simulated physiological conditions. Electrochemical impedance was measured every three days for the first two weeks post-implantation, and subsequently on a weekly or biweekly basis.

The 1 kHz impedance as a fast and common metric for assessing the implant and tissue reactions was first investigated. The results revealed distinct differences between the two configurations (FIG. 20). The μECoG control group exhibited significant impedance variability, characterized by three distinct phases with breakpoints on days 11 and 40, identified using a generalized additive model (GAM). The inventors observed an initial surge of impedance followed by a gradual decrease and recovery to baseline. Specifically, μECoG electrode exhibited a sharp increase from an initial value of ~20 kΩ on day 1 to ~540 kΩ on day 11 (slope=55.8 kΩ d⁻¹, p<0.001), followed by a sharp decrease to ~74 kΩ on day 40 (slope=−12.9 kΩ d⁻¹, p<0.001), and then decreased slowly (slope=−0.4 kΩ d⁻¹, p<0.001). The impedance fluctuations observed in this study reflect dynamic biological processes at the electrode-tissue interface following implantation and are consistent with previous studies. The initial increase in impedance is driven by biofouling and protein adsorption, followed by a gradual decrease indicative of protein densification and tissue remodeling. By day 40, the impedance stabilized at 70.4±1.2 kΩ (slope=−0.4, p<0.001), suggesting that the reactive tissue response had reached equilibrium. Histological analysis supports these findings, showing a rapid initial increase in glial cell density at the implantation site, followed by stabilization marked by reduced glial cell activation and formation of tissue encapsulation. Although the impedance recovered to levels close to baseline, it remained slightly elevated, indicating persistent changes at the electrode-tissue interface.

In contrast, aGel-μECoG electrodes demonstrated remarkable stability throughout the implantation period, with impedance consistently remaining within a narrow range (32.3±1.1 kΩ), indicating a more stable electrode-tissue interface. Histological and SHG imaging revealed minimal fibrotic capsule formation in the arachnoid and explanted devices.

While single-frequency impedance measurements provide a convenient metric for evaluating electrode functionality, wide-frequency spectrum EIS combined with equivalent circuit modeling offers a more comprehensive understanding of tissue reactions at the electrode-tissue interface. Combing EIS with circuit modeling (FIGS. 21, 22A and 22B), it was found that the ECoG group exhibited a significant decrease in the constant phase element (Z_{CPE}) exponent term a and capacitance magnitude in the Z_{CPE} (FIG. 23), indicating a transition toward more resistive characteristics at the interface. By contrast, the Z_{CPE} exponent term a. and capacitance magnitude in the aGel-μECoG group remained stable for two months. Additionally, the tissue resistance R_{T} increased rapidly in both groups during the acute phase (<2 weeks), but the μECoG stabilized at two orders of magnitude higher impedance than that of the aGel-μECoG group (~1000 kΩ vs. ~10 kΩ) (FIG. 24), due to the formation of less conductive encapsulation layer. Furthermore, the smaller tissue capacitance (C_{T}) in the μECoG group after stabilization also suggested the formation of a thicker encapsulation layer, which is consistent with the histological analysis (FIG. 18). These results reinforce the findings that aGel-μECoG preserves a stable electrode-tissue interface in vivo.

### Example 6. Long-Term Signal Stability of aGel-μECoG

Electrophysiological signals with well-defined patterns, such as visual evoked potentials, auditory evoked potentials, sensory evoked potentials, and epileptiform signals, are commonly used to evaluate the chronic stability of implanted surface arrays. Among these, steady-state visual evoked potentials (SSVEP) provide a non-invasive and reliable measure of cortical activity owing to their consistent responses and minimal training requirements. SSVEPs elicit widespread activation of the visual cortex, ensuring uniform SNRs across the electrodes. Additionally, the SNR of the SSVEP is quantified in the frequency domain and is thus not susceptible to environmental noise.

Similar to the long-term evaluation of impedance, the 64-channel aGel-μECoG and μECoG were implanted in the left visual cortex of rats. The rats were head-fixed and awake during recordings, with an 8-Hz flashing light positioned 10 cm in front of their eyes. Light stimuli were delivered at a constant intensity with a 50% duty cycle to ensure consistent activations. The SSVEP signals were recorded weekly for the first 12 weeks and biweekly thereafter for up to 16 weeks. Representative results from an aGel-μECoG electrode showed stable signal amplitude and power spectra over 16 weeks, with periodic neural activity characterized by distinct peaks at 8 Hz and their corresponding harmonics. When the rat's eyes were covered, these frequency-specific signals, particularly even harmonics (e.g., 16 Hz and 32 Hz), disappeared, confirming that the visual cortex was the origin of the recorded activity.

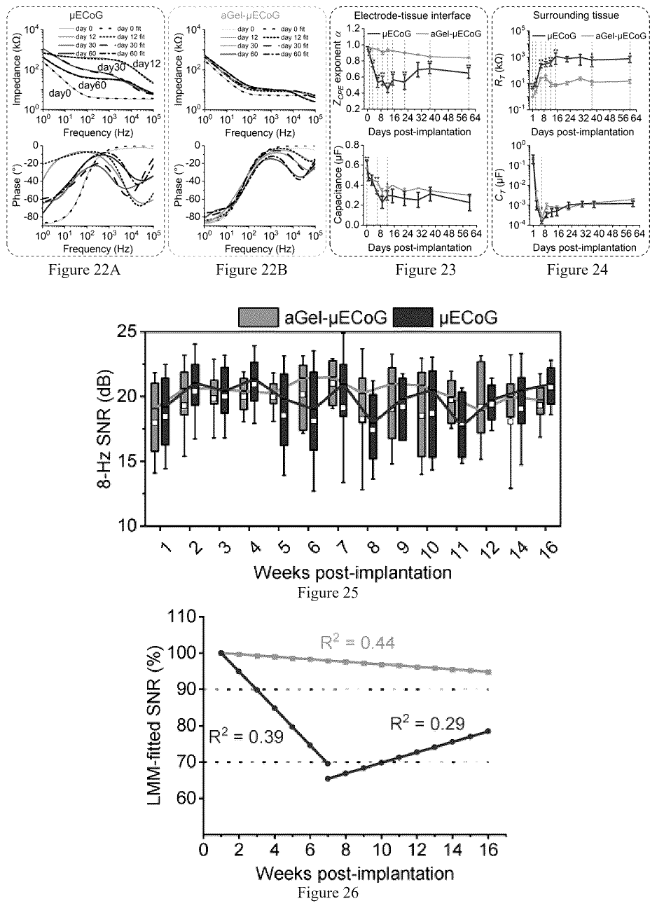

To assess the signal stability over time, the SNR at 8 Hz was analyzed in five rats for each device type over a 16-week period (FIG. 25). Signal stability analysis is challenging because of potential variabilities in the electrode-tissue interface, individual subject differences, and attention and physiological status of the animals. To address these confounding factors, a linear mixed-effects model (LMM) was employed, incorporating electrode type (ECoG vs. aGel-μECoG), post-implantation time, and their interaction as fixed effects, while accounting for unintended variations as random effects.

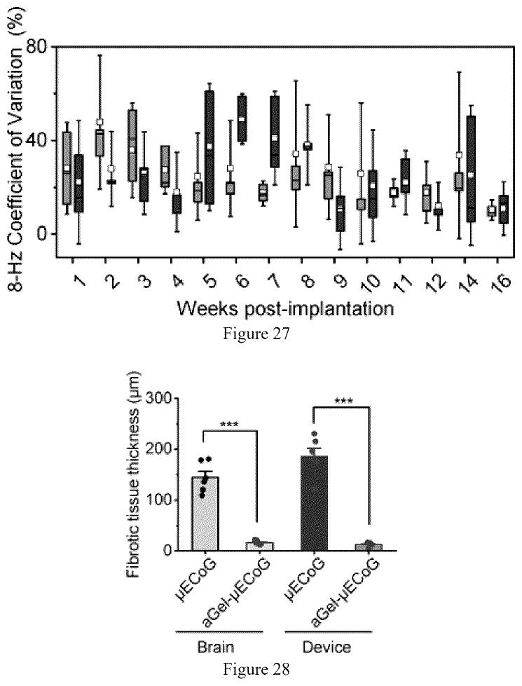

LMM modeling showed that both μECoG and aGel-μECoG had similar baseline signal quality (β₁=10.3, p=0.594; Table S4), corroborating our previous findings that a 10-μm aGel coating does not change signal quality. The aGel-μECoG electrodes maintained a consistent trend over time (β₂=−0.4, p=0.029), with a predicted SNR of 94.8% at 16 weeks post-implantation. The μECoG arrays exhibited a significantly different SNR trend over time compared with the aGel-ECoG arrays (β₃=−1.0, p<0.001). It was found that the μECoG exhibited a nonlinear SNR trend characterized by an initial decrease, followed by an increase. Using the minimum mean square error method, the data were segmented into two periods: before and after week 7. The analysis revealed a significant SNR decrease before week 7 (p<0.001, predicted SNR at week 7=69.5%) and a subsequent significant increase after week 7 (p<0.001, predicted SNR at week 7=65.4%, at week 16=78.4%; FIG. 26). Furthermore, intra-electrode consistency analysis showed that aGel-μECoG electrodes exhibited significantly lower coefficients of variation for an 8-Hz SNR (p <0.001, two-way ANOVA, FIG. 27), indicating reduced SNR variability and a more homogeneous electrode-tissue interface. The observed fluctuations and variabilities in signal quality for the μECoG group are likely driven by dynamic biological reactions at the electrode-tissue interface. While the impedance begins to decrease after day 11, the decline in SNR persists until week 7, indicating a decoupling between these two metrics. These findings suggest that local changes in tissue resistivity, driven by inflammation and encapsulation, are the potential dominant factors influencing signal quality, which are consistent with previous studies.

At 16 weeks post-implantation, brain tissues were examined using H&E staining, and explanted devices were evaluated using bright-field imaging, propidium iodide staining, and SHG imaging. The μECoG devices showed opaque, thick, and uneven fibrous tissue encapsulation under bright-field imaging, with SHG imaging indicating strong collagen accumulation around the implants. Propidium iodide staining of the extracted electrodes showed significantly enhanced cellular encapsulation on μECoG, which correlated with the SHG results. H&E imaging revealed the formation of a thick fibrotic capsule beneath the μECoG on the brain slices. By contrast, aGel-ECoG remained transparent, with no detectable fibrotic capsules on either the device or the brain surface. Statistical analysis of H&E staining and propidium iodide staining revealed significantly thicker fibrotic capsules beneath the μECoG implants on both the brain surface (FIG. 28, left) and device surface (FIG. 28, right) than those of aGel-μECoG. This nonhomogeneous fibrotic capsule may be the primary cause of signal fluctuations and increased variability across μECoG arrays. The findings indicate that while both electrode types demonstrate similar baseline performance, aGel-μECoG electrodes exhibit enhanced longitudinal stability and spatial consistency during chronic implantation. This underscores their suitability for long-term neural recording applications.

### Discussion

Subdural μECoG arrays offer high spatial and temporal resolutions with superior signal quality, making them valuable tools for neural interfacing. However, their long-term implantation often leads to neuroinflammation and fibrotic tissue growth at the electrode-tissue interface, leading to progressive signal deterioration. Recent advances have highlighted the potential of hydrogel-based functional interfaces in bioelectronics due to their inherent biocompatibility, mechanical compliance, and bioadhesiveness. The present disclosure provides as an example an aGel-μECoG platform that integrates a flexible, high-density μECoG array with an ultrathin PVA/PTPM hydrophilic/hydrophobic heteronetwork. The resulting hydrogel demonstrated excellent swelling resistance, mechanical compliance (Young's modulus of ~109.4±19.5 kPa), and robust bioadhesiveness (~25.2±3.8 kPa). Unlike chemically bonded adhesive strategies that pose potential risks of chemical contamination and tissue damage, the present disclosure employed a dry cross-linked hydrogen bonding mechanism. This approach enabled a strong yet reversible bond to the delicate brain surface, ensuring mechanical stability while minimizing tissue damage.

The inventors systematically investigated the hydrogel's impact on signal communication and optimized its design parameters for high electrical fidelity. High-precision functional cortical mapping experiments confirmed that the optimized aGel-μECoG preserved weak signal amplitude and maintained submillimeter spatial resolution. Long-term implantation studies demonstrated its superior ability to mitigate neuroinflammation and suppress fibrotic tissue formation, resulting in a 20-fold reduction in acute-phase impedance increases compared to conventional μECoG arrays. Furthermore, aGel-μECoG group maintained stable SSVEP recordings, with a signal-to-noise ratio of 94.8% over 16 weeks, significantly outperforming conventional μECoG arrays.

While the foregoing illustrates and describes exemplary embodiments of this invention, it is to be understood that the invention is not limited to the construction disclosed herein. The invention can be embodied in other specific forms without departing from the spirit or essential attributes.

What is claimed is:

1. An implantable bioelectronic device, comprising:
a flexible insulating substrate;
a conductive circuit layer disposed on the flexible insulating substrate and comprising an array of electrodes comprising one or more electrodes;
an insulating encapsulation layer encapsulating the conductive circuit layer and comprising an opening, wherein the electrodes are exposed through the opening; and
a conductive adhesive interface layer disposed on the insulating encapsulation layer and comprising an exposed surface and a filling portion that extends into the opening, wherein the exposed surface is electrically connected to the electrodes through the filling portion, the conductive adhesive interface layer comprises a hydrophilic polymer and a hydrophobic polymer, and wherein the hydrophobic polymer comprises a polymerized network of silane-based monomers.

2. The device according to claim 1, wherein:
the silane-based monomers each independently has a general formula: Rₐ(CH₂)ₙSi(OR_{b})₃,
Rₐ represents a group comprising a polymerizable functional group selected from the group consisting of an addition-polymerizable functional group, a condensation-polymerizable functional group, and a ring-opening polymerizable functional group;
R_{b}s are the same or different, and are each independently selected from the group consisting of C₁₋₁₂ alkyl, and —C₁₋₁₂ alkylene-O—C₁₋₁₂ alkyl; and
n is an integer of 0 to 12;
preferably, the silane-based monomers comprise: 3-(trimethoxysilyl)propyl methacrylate (TPM), vinyltrimethoxysilane (KH171), vinyltris (β-methoxyethoxy) silane (KH172), vinyltriethoxysilane (KH151), glycidoxypropyltrimethoxysilane, or combinations thereof; more preferably, the silane-based monomers are 3-(trimethoxysilyl)propyl methacrylate.

3. The device according to claim 1, wherein:
the hydrophilic polymer has a plurality of hydrophilic groups and is configured such that, when the conductive adhesive interface layer is put in contact with a moist biological tissue, the hydrophilic groups form multiple hydrogen bonds with nitrogen-containing or oxygen-containing functional groups of the biological tissue; and/or
the hydrophilic groups comprise one or more hydroxyl groups, one or more carboxyl groups, one or more amino groups, and/or one or more sulfonic acid groups; and/or
the hydrophilic polymer is selected from the group consisting of: polyvinyl alcohol (PVA), polyethylene oxide, hydrophilic polyurethane, hydrophilic epoxy resin, polyacrylamide, polyethylene glycol, poly(hydroxyethyl methacrylate), poly(hydroxyethyl acrylate), polyacrylic acid, copolymers thereof, and combinations thereof.

4. The device according to claim 1, wherein the conductive adhesive interface layer is formed by in-situ polymerization of a composition comprising the hydrophilic polymer or monomers thereof and the silane-based monomers, particularly within the opening;
preferably, the composition comprises 2-15 parts by weight of the hydrophilic polymer or monomers thereof, 0.1-10 parts by weight of the silane-based monomers, and 0.0001-0.5 parts by weight of an initiator.

5. The device according to claim 1, wherein:
the mass ratio of the hydrophilic polymer to the hydrophobic polymer ranges from 3.5:1 to 17:1; and/or
the conductive adhesive interface layer is in a form of hydrogel or dehydrated hydrogel; and/or
the conductive adhesive interface layer is a hydrogel and has a thickness of about 1~50 μm; and/or
the conductive adhesive interface layer is a hydrogel and has an electrical conductivity of about 0.5~10 S/m; and/or
the conductive adhesive interface layer is a hydrogel and has a Young's modulus of about 60~150 kPa; and/or
the conductive adhesive interface layer is a hydrogel and has an adhesion strength to moist biological tissue of about 20~30 kPa; and/or
the conductive adhesive interface layer is a hydrogel and has a peel strength to moist biological tissue of 20~35 Pa; and/or
the conductive adhesive interface layer is a hydrogel and has an adhesion strength to the insulating encapsulation layer of 100~150 kPa; and/or
the conductive adhesive interface layer is a hydrogel and has an average light transmittance in the visible light range greater than 85%, wherein the wavelength of visible light is 400-800 nm; and/or
after swelling in water for 60 minutes, the mass swelling ratio of the conductive adhesive interface layer is 86±11%; and/or
after swelling in water for 60 minutes, the volume swelling ratio of the conductive adhesive interface layer is 133±17%.

6. The device according to claim 1, wherein:
the flexible insulating substrate comprises at least one of glass, silicon, polyimide, polycarbonate, perylene, polypropylene, polymethyl methacrylate (PMMA), polyethylene terephthalate (PETE), polydimethylsiloxane (PDMS), polyurethane, styrene-ethylene-butylene-styrene (SEBS), and combinations thereof, and/or
the thickness of the flexible insulating substrate is selected from 1 to 100 micrometers; and/or
the insulating encapsulation layer comprises at least one of glass, silicon, polyimide, polycarbonate, perylene, polypropylene, polymethyl methacrylate (PMMA), polyethylene terephthalate (PETE), polydimethylsiloxane (PDMS), polyurethane, styrene-ethylene-butylene-styrene (SEBS), and combinations thereof, and/or
the thickness of the insulating encapsulation layer is selected from 1 to 100 micrometers.

7. The device according to claim 1, further comprising:
a conductive polymer layer located between the electrode and the filling portion;
preferably, the conductive polymer layer comprises a material selected from the group consisting of poly(3,4-ethylenedioxythiophene) (PEDOT), poly(3,4-ethylenedioxythiophene) polystyrene sulfonate (PEDOT:PSS), polythiophene, poly(p-phenylene sulfide), polypyrrole (PPy), polycarbazole, polyindole, polyazepine, polyaniline (PAni), and combinations thereof.

8. The device according to claim 1, wherein the conductive circuit layer comprises:
a first metal layer layered on a surface of the flexible insulating substrate; and
a second metal layer layered on a side of the first metal layer away from the flexible insulating substrate,
wherein the first metal layer comprises a metal different from that comprised in the second metal layer, and
wherein the electrode is formed on or is part of the second metal layer;
preferably, the first metal layer comprises titanium, and/or the second metal layer comprises gold; and/or the first metal layer is made of titanium, and/or the second metal layer is made of gold.

9. A method for preparing an implantable bioelectronic device, comprising:
providing a semi-encapsulated substrate, wherein the semi-encapsulated substrate comprises: a flexible insulating substrate; a conductive circuit layer disposed on the flexible insulating substrate and comprising an array of electrodes comprising one or more electrodes; and an insulating encapsulation layer encapsulating the conductive circuit layer and comprising an opening, wherein the electrodes are exposed through the opening; and
forming a conductive adhesive interface layer on the insulating encapsulation layer, wherein the conductive adhesive interface layer comprises an exposed surface and a filling portion that extends into the opening, wherein the exposed surface is electrically connected to the electrodes through the filling portion, and wherein the conductive adhesive interface layer comprises a hydrophilic polymer and a hydrophobic polymer, and wherein the hydrophobic polymer comprises a polymerized network of silane-based monomers.

10. The method according to claim 9, wherein the step of forming the conductive adhesive interface layer comprises:
providing a solution comprising the hydrophilic polymer or monomers thereof in an organic solvent; preferably, the organic solvent is selected from the group consisting of: dimethyl sulfoxide, N,N-dimethylformamide, chloroform, tetrahydrofuran, toluene, ethanol, and ethylene glycol, and combinations thereof;
mixing the solution with an initiator and the silane-based monomers to form a pre-polymerization solution; preferably, the initiator is an oil-soluble thermal initiator or a photoinitiator; preferably, the oil-soluble thermal initiator is selected from the group consisting of: azobisisobutyronitrile, benzoyl peroxide, and combinations thereof, preferably, the photoinitiator is selected from the group consisting of: 2,2-dimethoxy-2-phenylacetophenone (DMPA), 2-hydroxy-2-methyl-1-phenyl-1-propanone, 1-hydroxycyclohexyl phenyl ketone, 2-methyl-1-[4-(methylthio)phenyl]-2-morpholinopropan-1-one, phenylbis(2,4,6-trimethylbenzoyl)phosphine oxide, 1-hydroxycyclohexyl phenyl ketone, 2-hydroxy-methylphenylpropan-1-one, benzoin dimethyl ether, and combinations thereof;
adding the pre-polymerization solution onto a surface of the insulating encapsulation layer and into the opening(s); and
subjecting the initiator to initiation conditions to form the conductive adhesive interface layer from the pre-polymerization solution;
preferably, in parts by weight, the pre-polymerization solution comprises: hydrophilic polymer 2-15 parts, silane-based monomer 0.1-10 parts, initiator 0.0001-0.5 parts, organic solvent 20-70 parts; preferably, in parts by weight, the pre-polymerization solution comprises: hydrophilic polymer 3-10 parts, silane-based monomer 0.15-4 parts, initiator 0.015-0.04 parts, organic solvent 30-60 parts.

11. The method according to claim 9, wherein:
the silane-based monomers each independently has a general formula: Rₐ(CH₂)ₙSi(OR_{b})₃,
Rₐ represents a group comprising a polymerizable functional group selected from the group consisting of an addition-polymerizable functional group, a condensation-polymerizable functional group, and a ring-opening polymerizable functional group;
R_{b}s are the same or different, and are each independently selected from the group consisting of C₁₋₁₂ alkyl, and —C₁₋₁₂ alkylene-O—C₁₋₁₂ alkyl; and
n is an integer of 0 to 12;
preferably, the silane-based monomers comprise: 3-(trimethoxysilyl)propyl methacrylate (TPM), vinyltrimethoxysilane (KH171), vinyltris (β-methoxyethoxy) silane (KH172), vinyltriethoxysilane (KH151), glycidoxypropyltrimethoxysilane, and combinations thereof, more preferably, the silane-based monomer is 3-(trimethoxysilyl)propyl methacrylate (TPM).

12. The method according to claim 9, wherein the hydrophilic polymer is selected from the group consisting of: polyvinyl alcohol (PVA), polyethylene oxide, hydrophilic polyurethane, hydrophilic epoxy resin, polyacrylamide, polyethylene glycol, poly(hydroxyethyl methacrylate), poly(hydroxyethyl acrylate), polyacrylic acid, copolymers thereof, and combinations thereof.

13. The method according to claim 9, wherein the mass ratio of the hydrophilic polymer or monomers thereof to the hydrophobic polymer ranges from 3.5:1 to 17:1.

14. The method according to claim 9, wherein the method further comprises:
a solvent exchange step in an aqueous solution to eliminate the organic solvent in the conductive adhesive interface layer; and/or
a dehydration step to dry the conductive adhesive interface layer.

15. A human-machine interface system, comprising the implantable bioelectronic device according to claim 1.

16. Use of a silane-based monomer having a general formula: Rₐ(CH₂)ₙSi(OR_{b})₃ for preparing a conductive adhesive interface layer on an implantable electrode, wherein the conductive adhesive interface layer is capable of: reducing neuroinflammation; reducing fibrotic tissue growth at the electrode-tissue interface; preserving electrical signal fidelity; and/or maintaining the electrode in position, and
wherien Rₐ represents a group comprising a polymerizable functional group selected from the group consisting of an addition-polymerizable functional group, a condensation-polymerizable functional group, and a ring-opening polymerizable functional group;
R_{b}s are the same or different, and are each independently selected from the group consisting of C₁₂ alkyl, and —C₁₋₁₂ alkylene-O—C₁₋₁₂ alkyl; and
n is an integer of 0 to 12.

17. The use according to claim 16, wherein:
the conductive adhesive interface layer comprises a hydrophilic polymer and a hydrophobic polymer formed by polymerization of a plurality of the silane-based monomers; and/or
the implantable electrode is for providing an electric field, or an electrical stimulation to or detecting a biological signal of a subject carrying the implantable electrode, preferably, the biological signal comprises an electrocorti cogram (ECoG) signal, an electroencephalogram (EEG) signal, an electromyogram (EMG) signal, a cardiac electrogram signal, or an evoked compound action potential (ECAP) signal.

18. The use according to claim 17, wherein:
the silane-based monomers comprise: 3-(trimethoxysilyl)propyl methacrylate (TPM), vinyltrimethoxysilane (KH171), vinyltris (β-methoxyethoxy) silane (KH172), vinyltriethoxysilane (KH151), glycidoxypropyltrimethoxysilane, or combinations thereof; preferably, the silane-based monomer is 3-(trimethoxysilyl)propyl methacrylate (TPM); and/or
the hydrophilic polymer is selected from the group consisting of: polyvinyl alcohol (PVA), polyethylene oxide, hydrophilic polyurethane, hydrophilic epoxy, polyacrylamide, polyethylene glycol, polyhydroxy ethyl methacrylate, polyhydroxy ethyl acrylate, poly acrylic acid, copolymers thereof, and combinations thereof, and/or
the mass ratio of the hydrophilic polymer to the hydrophobic polymer ranges from 3.5:1 to 17:1.

19. A method for diagnosing, treating or preventing a disease in a subject in need thereof, comprising implanting the implantable bioelectronic device according to claim 1 into a tissue of the subject.

20. The method according to claim 19, wherein:
the implantable bioelectronic device comprises dehydrated conductive adhesive interface layer; and/or
the tissue is selected from the group consisting of brain, muscle and heart; and/or
the disease is selected from the group consisting of epilepsy, sleep and mental disorders, stroke, brain, spinal and peripheral nerve injuries, and neurodegenerative diseases.
